(19) United States
(12) Patent Application Publication
(10) Pub. No.: US 2025/0174358 A1
(43) Pub. Date: May 29, 2025

(54) METHODS AND SYSTEMS FOR CLASSIFICATION OF DISEASE ENTITIES VIA MIXTURE MODELING

(71) Applicant: Foundation Medicine, Inc., Boston, MA (US)

(72) Inventors: James HABERBERGER, Huntsville, NC (US); Shakti H. RAMKISSOON, Raleigh, NC (US)

(73) Assignee: Foundation Medicine, Inc., Boston, MA (US)

(21) Appl. No.: 18/965,698

(22) Filed: Dec. 2, 2024

Related U.S. Application Data
(63) Continuation of application No. PCT/US2023/023612, filed on May 25, 2023.
(60) Provisional application No. 63/348,892, filed on Jun. 3, 2022.

(51) Int. Cl. G16H 50/20; G16B 20/00; G16B 40/20

(52) U.S. Cl. CPC G16H 50/20; G16B 20/00; G16B 40/20

(57) ABSTRACT

Methods for identifying disease subgroups are described. The methods may comprise, for example, receiving subject data for a plurality of subjects diagnosed with the disease; creating a plurality of candidate best fit latent class or mixture models by: i) providing an estimate of a number of subgroups; ii) generating a set of models, each model of the set comprising the same estimate of the number of subgroups; iii) selecting a candidate best fit model from the set; and iv) repeating (i)-(iii) at least once using a different estimate of the number of subgroups to obtain a plurality of candidate best fit models; selecting a best fit model from the plurality of candidate best fit models based on a fit statistic; and applying the best fit model to the subject data to identify a number of subgroups for the disease and an associated genomic profile for each subgroup.

### CROSS-REFERENCE TO RELATED APPLICATIONS

This application is a continuation application of International Application No. PCT/US2023/023612, filed internationally on May 25, 2023, which claims the priority benefit of U.S. Provisional Patent Application Ser. No. 63/348,892, filed Jun. 3, 2022, the contents of each of which are incorporated herein by reference in their entirety.

### FIELD OF THE INVENTION

The present disclosure relates generally to methods and systems for analyzing genomic profiling data, and more specifically to methods and systems for identifying disease subgroups based on genomic profiling data.

### BACKGROUND

Genetic disorders are diseases or conditions caused by one or more genomic alterations (e.g., changes in the DNA sequence relative to the normal DNA sequence). Some genetic disorders, for example, are caused by a mutation in a single gene (i.e., monogenic disorders). However, many genetic disorders are caused by mutations in multiple genes and/or chromosomes (i.e., polygenic disorders), or by mutations in multiple genes and/or chromosomes in combination with a variety of other lifestyle or environmental factors (i.e., multifactorial disorders).

In some cases, genomic alterations may be inherited from one or both parents, and are thus present in an individual at birth. Such genomic alterations may, in some instances, give rise to a hereditary disorder in the individual (e.g., an inherited monogenic or polygenic disease). In other cases, genomic alterations may be acquired either randomly or due to exposure to an environmental factor over the course of an individual's life. Such genomic alterations may, in some instances, give rise to an acquired disorder in the individual (e.g., an acquired monogenic or polygenic disease). In yet other cases, genomic alterations in multiple genes and/or chromosomes, in combination with one or more additional factors (such as nutrition, lifestyle, alcohol or tobacco use, exposure to pollutants, etc.), may give rise to a multifactorial inherited or acquired disorder in an individual (e.g., a multifactorial inherited disease or a multifactorial acquired disease). The underlying genomic landscape for disease is thus complex, with many potential underlying genomic alterations that may act alone or in concert with one or more environmental factors to trigger disease.

Recent advances in comprehensive genomic profiling (CGP) have enabled clinicians to probe an array of potentially actionable, disease-defining genomic alterations. Combining CGP data with existing histopathological classification can provide an “integrated diagnosis” that adds depth to disease ontologies, and may facilitate clinical decision-making to improve healthcare outcomes. However, given the extremely varied genomic landscape of disease, methods for categorizing patients according to disease subgroups based on genomic data (alone, or in combination with other clinical data) and for identifying driving alterations in these disease subgroups may be crucial to identifying promising new therapeutic modalities, but continue to be challenging.

### BRIEF SUMMARY OF THE INVENTION

Disclosed herein are methods and systems for classification of disease entities to identify disease subgroups based on genomic data (alone, or in combination with other clinical data) for a cohort of patients that have been diagnosed with the disease, and to identify genomic profiles and driver mutations associated with each disease subgroup. The methods may comprise, for example, processing data for a plurality of patients diagnosed with a disease, where the patient data (e.g., patient genomic data and/or patient characteristics such as age, gender, height, weight, etc.) comprises an indication of whether a biomarker associated with the disease is present in each patient of the plurality; generating a plurality of candidate best fit models based on the subject data by iteratively estimating the number of disease subgroups to be identified, generating a set of models based on the current estimate of the number of disease subgroups to be identified, selecting a candidate best fit model from the set of models, and repeating these steps using a different estimate of the number of disease subgroups; selecting a best fit model from the plurality of candidate best fit models based on a fit statistic; and applying the best fit model to the patient data to identify a number of subgroups for the disease and an associated genomic profile for each subgroup.

The disclosed methods and systems provide advantages over previous approaches in terms of the ability to successfully categorize patients according to disease subgroups that are associated with, e.g., distinct driver mutations. Furthermore, the data modeling is more instructive for describing actionable and/or prognostic features for different disease subgroups. The disclosed methods for categorizing patients according to disease subgroups and identifying driver alterations in different disease subgroups has the potential to provide greater insight into patient disease characteristics and treatment options, facilitate treatment decisions by healthcare providers, improve treatment outcome predictions, and to facilitate the design of clinical trials and development of new therapeutics. In some embodiments, the disclosed methods and systems may also enable the use of smaller bait sets for complete genomic profiling assays, reduce the costs associated with sequencing nucleic acid samples derived from patients, and reduce the amount of patient sequencing data that needs to be processed and stored.

Disclosed herein are methods comprising: providing a plurality of nucleic acid molecules obtained from a sample from each of a plurality of subjects; ligating one or more adapters onto one or more nucleic acid molecules from the plurality of nucleic acid molecules obtained from each sample; amplifying the one or more ligated nucleic acid molecules from the plurality of nucleic acid molecules obtained from each sample; capturing amplified nucleic acid molecules from the amplified nucleic acid molecules for each sample; sequencing, by a sequencer, the captured nucleic acid molecules for each sample to obtain a plurality of sequence reads that represent the captured nucleic acid molecules for each sample; receiving, at one or more processors, sequence read data for the plurality of sequence reads from each sample; receiving, at the one or more processors, subject data for a plurality of subjects diagnosed with the disease, wherein the subject data is based, at least in part, on the sequence read data for each sample and comprises an indication of whether a biomarker associated with a disease is present; obtaining, using the one or more processors, a plurality of candidate best fit models based on the subject data by: i) providing a predefined estimate of a number of subgroups; ii) generating a set of models based on the subject data, each model of the set comprising the same predefined estimate of the number of subgroups; iii) selecting a candidate best fit model from the set of models; and iv) repeating (i)-(iii) at least once using a different predefined estimate of the number of subgroups to obtain the plurality of candidate best fit models; selecting a best fit model from the plurality of candidate best fit models based on a fit statistic; and determining, using the one or more processors, a number of subgroups for the disease and an associated genomic profile for each subgroup based on the best fit model and the subject data.

In some embodiments, the method further comprises determining to which disease subgroup an individual subject belongs based on the individual subject's data and the best fit model. In some embodiments, the method further comprises providing a treatment recommendation or outcome prediction for the individual subject based on the disease subgroup to which the individual subject belongs. In some embodiments, the method further comprising identifying a subgroup of subjects for participation in a clinical study based on a disease subgroup to which the subgroup of subjects belong. In some embodiments, the method further comprising identifying one or more driver mutations for a disease subgroup based on the associated genomic profile for the subgroup. In some embodiments, the selecting of the best fit model is performed, at least in part, by the one or more processors. In some embodiments, the selecting of the best fit model is performed, at least in part, by a user.

In some embodiments, the plurality of subjects have been diagnosed to have cancer. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypercosinophilic syndrome, systemic mastocytosis, familiar hypercosinophilia, chronic eosinophilic leukemia, neuroendocrine cancers, or a carcinoid tumor.

In some embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myclofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the method further comprising treating the subject with an anti-cancer therapy. In some embodiments, the anti-cancer therapy comprises a targeted anti-cancer therapy.

In some embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-crzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some embodiments, the method further comprises obtaining the sample from each subject of the plurality. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule.

In some embodiments, amplifying nucleic acid molecules comprises performing a polymerase chain reaction (PCR) amplification technique, a non-PCR amplification technique, or an isothermal amplification technique.

In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencer comprises a next generation sequencer.

In some embodiments, one or more of the plurality of sequencing reads overlap one or more gene loci within one or more subgenomic intervals in the sample. In some embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In some embodiments, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAPI, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAFI, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCILI, WT1, XPO1, XRCC2, ZNF217, ZNF703, or any combination thereof.

In some embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In some embodiments, the method further comprises generating, by the one or more processors, a report indicating the number of identified disease subgroups and an associated genomic profile for each subgroup. In some embodiments, the method further comprises generating, by the one or more processors, a report indicating the disease subgroup to which an individual subject belongs and an associated genomic profile for the individual subject. In some embodiments, the method further comprises transmitting the report to a healthcare provider. In some embodiments, the report is transmitted via a computer network or a peer-to-peer connection.

Disclosed herein are methods for identifying a plurality of subgroups for a disease, the methods comprising: receiving, at one or more processors, subject data for a plurality of subjects diagnosed with the disease, wherein the subject data comprises an indication of whether a biomarker associated with the disease is present; obtaining, using the one or more processors, a plurality of candidate best fit models based on the subject data by: i) providing a predefined estimate of a number of subgroups; ii) generating a set of models based on the subject data, each model of the set comprising the same predefined estimate of the number of subgroups; iii) selecting a candidate best fit model from the set of models; and iv) repeating (i)-(iii) at least once using a different predefined estimate of the number of subgroups to obtain the plurality of candidate best fit models; selecting a best fit model from the plurality of candidate best fit models based on a fit statistic; and determining, using the one or more processors, a number of subgroups for the disease and an associated genomic profile for each subgroup based on the best fit model and the subject data.

Also disclosed herein are methods for identifying a plurality of subgroups for a disease, the methods comprising: receiving, by at least one processor, subject data for a plurality of subjects diagnosed with the disease; obtaining, by the at least one processor, a plurality of candidate best fit models based on the subject data by: i) providing a predefined estimate of a number of subgroups; ii) generating a set of models based on the subject data, each model of the set comprising the same predefined estimate of the number of subgroups; iii) selecting a candidate best fit model from the set of models; and iv) repeating (i)-(iii) at least once using a different predefined estimate of the number of subgroups to obtain the plurality of candidate best fit models; selecting a best fit model from the plurality of candidate best fit models based on a fit statistic; and determining, using the one or more processors, a number of subgroups for the disease and an associated genomic profile for each subgroup based on the best fit model and the subject data.

In some embodiments of any of the methods disclosed herein, the selecting of the best fit model is performed, at least in part, by the one or more processors. In some embodiments, the selecting of the best fit model is performed, at least in part, by a user.

In some embodiments, the method further comprises determining to which disease subgroup an individual subject belongs based on the individual subject's data and the best fit model. In some embodiments, the method further comprises providing a treatment recommendation or outcome prediction for the individual subject based on the disease subgroup to which the individual subject belongs. In some embodiments, the method further comprises identifying a subgroup of subjects for participation in a clinical study based on a disease subgroup to which the subgroup of subjects belong. In some embodiments, the method further comprises identifying one or more driver mutations for a disease subgroup based on the associated genomic profile for the subgroup. In some embodiments, a biomarker associated with the disease comprises one or more genetic mutations.

In some embodiments, the method further comprises modifying a specified panel of genes used for genomic profiling of the disease based on the identified number of subgroups for the disease and the associated genomic profile for each subgroup. In some embodiments, modification of the specified panel of genes comprises modifying a bait set used to generate genomic profile data. In some embodiments, the subject data for the plurality of subjects comprises genomic profile data. In some embodiments, the subject data for the plurality of subjects further comprises data regarding subject sex, subject age, subject gender, subject height, subject weight, subject clinical history, subject sample type, or any combination thereof. In some embodiments, the genomic profile data comprises a binary indication of whether a pathogenic mutation is present in each gene of a specified panel of genes. In some embodiments, the genomic profile data is received in the form of a matrix comprising M rows×N columns, where each of the M rows comprises data for an occurrence of a genetic mutation in a given gene of the specified panel of genes in an individual subject, and each of the N columns comprises genetic mutation data across the specified panel of genes for an individual subject of the plurality of subjects.

In some embodiments, the plurality of candidate best fit models comprise non-probabilistic models. In some embodiments, the plurality of candidate best fit models comprise probabilistic models. In some embodiments, the predefined estimate of the number of subgroups includes a number of subgroups identified using a variational Bayesian method or agglomerative clustering method. In some embodiments, the probabilistic models of a set of probabilistic models comprising the same predefined estimate of the number of subgroups are generated using different sets of initialization parameters. In some embodiments, the different sets of initialization parameters comprise sets of randomly assigned initialization parameters. In some embodiments, the different sets of initialization parameters comprise initialization parameters generated using a k-nearest neighbor (KNN) method.

In some embodiments, the candidate best fit model from the set of models comprising the same predefined estimate of the number of subgroups is selected based on optimization of an objective function. In some embodiments, the candidate best fit model from the set of models comprising the same predefined estimate of the number of subgroups is selected based on optimization of a log-likelihood function. In some embodiments, the selected candidate best fit model is the model which has a maximum log-likelihood value. In some embodiments, the candidate best fit model is selected by the one or more processors at least in part based on a previous user selection. In some embodiments, the candidate best fit model is selected by a machine learning model trained at least in part based on previous user selections.

In some embodiment, the method further comprises specifying a set of one or more operational parameters for generating the set of models comprising the same predefined estimate of the number of subgroups, wherein the specified operational parameters comprise a maximum number of epochs to be performed, a tolerance on log-likelihood function for each epoch, a metric for calculation of covariance for each epoch, or any combination thereof.

In some embodiments, models of the set of models comprising the same predefined estimate of the number of subgroups are generated using a latent class analysis, clustering technique, or mixture modeling technique. In some embodiments, the models are generated using a clustering technique, and wherein the clustering technique comprises a k-means clustering technique, a mean-shift clustering technique, a density-based spatial clustering of applications with noise (DBSCAN) technique, an agglomerative hierarchical clustering technique, a random forest technique, or any combination thereof. In some embodiments, the clustering technique comprises a k-means clustering technique, and the output of the model comprises, for each subgroup (cluster), a centroid, an intra-cluster distance, an inertia value, an inter-cluster distance for each pair of clusters, a Dunn index, an expected prevalence for each cluster, or any combination thereof. In some embodiments, the models are generated using a mixture model technique, and wherein the mixture modeling technique comprises a Gaussian mixture modeling technique, a multivariate Gaussian mixture modeling technique, a categorical mixture modeling technique, a variational Bayes mixture modeling technique, or any combination thereof. In some embodiments, the mixture modeling technique comprises a Gaussian mixture modeling technique, and the output of the probabilistic model comprises, for each subgroup, a mean, μ, a covariance, Σ, and a mixing probability, π, an expected prevalence, or any combination thereof. In some embodiments, determining optimal values for one or more model parameters comprises determining a maximum likelihood estimate of the one or more model parameters.

In some embodiments, the predefined estimate of the number of subgroups ranges from 1 to 100. In some embodiments, the predefined estimate of the number of subgroups ranges from 1 to 50. In some embodiments, the predefined estimate of the number of subgroups ranges from 1 to 20.

In some embodiments, the set of models generated for each predefined estimate of the number of subgroups comprises from 2 to 10 models.

In some embodiments, steps (i)-(iii) are repeated at least 2 times using a different predefined estimate of the number of subgroups each time. In some embodiments, steps (i)-(iii) are repeated at least 5 times using a different predefined estimate of the number of subgroups each time. In some embodiments, steps (i)-(iii) are repeated at least 10 times using a different predefined estimate of the number of subgroups each time.

In some embodiments, the fit statistic comprises an Akaike Information Criterion (AIC) score, a Bayesian Information Criterion (BIC) score, a Calinski-Harabasz (CH) score, or any combination thereof.

In some embodiments, the disease comprises a multifactorial inherited disorder. In some embodiments, the multifactorial inherited disorder comprises heart disease, high blood pressure, Alzheimer's disease, arthritis, diabetes, cancer, obesity, or any combination thereof. In some embodiments, the disease comprises a cancer. In some embodiments, the specified panel of genes comprises between 10 and 100 genes for which there are known pathogenic or likely pathogenic mutations.

In some embodiments, the determination of to which disease subgroup an individual subject belongs is used to diagnose or confirm a diagnosis of disease in the subject. In some embodiments, the method further comprises selecting an anti-cancer therapy to administer to the subject based on the determination of to which disease subgroup the individual subject belongs. In some embodiments, the method further comprises determining an effective amount of an anti-cancer therapy to administer to the subject based on the determination of to which disease subgroup the individual subject belongs. In some embodiments, the method further comprises administering the anti-cancer therapy to the subject based on the determination of to which disease subgroup the individual subject belongs. In some embodiments, the anti-cancer therapy comprises chemotherapy, radiation therapy, immunotherapy, a targeted therapy, or surgery.

Disclosed herein are methods for diagnosing a disease, the methods comprising: diagnosing that a subject has the disease based on a determination of to which disease subgroup the subject belongs, wherein the disease subgroup is determined according to any of the methods described herein.

Also disclosed herein are methods for refining a diagnosis of disease, the methods comprising: refining a diagnosis that a subject has the disease based on a determination of to which disease subgroup the subject belongs, wherein the disease subgroup is determined according to any of the methods described herein.

Disclosed herein are methods of selecting an anti-cancer therapy, the methods comprising: responsive to a determination of to which cancer subgroup a subject belongs based on an analysis of a sample from a subject, selecting an anti-cancer therapy for the subject, wherein the determination of cancer subgroup is made according to any of the methods described herein.

Disclosed herein are methods of treating a cancer in a subject, comprising: responsive to a determination of to which cancer subgroup the subject belongs based on an analysis of a sample from the subject, administering an effective amount of an anti-cancer therapy to the subject, wherein the determination of cancer subgroup is made according to any of the methods described herein.

Disclosed herein are methods for monitoring cancer progression or recurrence in a subject, the methods comprising: determining to which cancer subgroup a subject belongs based on an analysis of a first sample obtained from the subject at a first time point according to any of the methods described herein; determining to which cancer subgroup a subject belongs based on an analysis of a second sample obtained from the subject at a second time point; and comparing the first determination of cancer subgroup to the second determination of cancer subgroup, thereby monitoring the cancer progression or recurrence. In some embodiments, the second determination of cancer subgroup is determined according to any of the methods described herein. In some embodiments, the method further comprises selecting an anti-cancer therapy for the subject in response to the cancer progression. In some embodiments, the method further comprises administering an anti-cancer therapy to the subject in response to the cancer progression. In some embodiments, the method further comprises adjusting an anti-cancer therapy for the subject in response to the cancer progression. In some embodiments, the method further comprises adjusting a dosage of the anti-cancer therapy or selecting a different anti-cancer therapy in response to the cancer progression. In some embodiments, the method further comprises administering the adjusted anti-cancer therapy to the subject. In some embodiments, the first time point is before the subject has been administered an anti-cancer therapy, and wherein the second time point is after the subject has been administered the anti-cancer therapy. In some embodiments, the subject has a cancer, is at risk of having a cancer, is being routine tested for cancer, or is suspected of having a cancer. In some embodiments, the cancer is a solid tumor. In some embodiments, the cancer is a hematological cancer. In some embodiments, the anti-cancer therapy comprises chemotherapy, radiation therapy, immunotherapy, a targeted therapy, or surgery.

In some embodiments of any of the methods described herein, the method further comprises determining to which disease subgroup an individual subject belongs based on an analysis of a sample obtained from the individual subject as a diagnostic value associated with the sample. In some embodiments, the method further comprises generating a genomic profile for the subject based on the determination of to which disease subgroup the subject belongs. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test. In some embodiments, the method further comprises selecting an anti-cancer therapy, administering an anti-cancer therapy, or applying an anti-cancer therapy to the subject based on the generated genomic profile. In some embodiments, the determination of cancer subgroup for the sample is used in making suggested treatment decisions for the subject. In some embodiments, the determination of cancer subgroup for the sample is used in applying or administering a treatment to the subject.

Disclosed herein are systems comprising: one or more processors; and a memory communicatively coupled to the one or more processors and configured to store instructions that, when executed by the one or more processors, cause the system to: receive subject data for a plurality of subjects diagnosed with a disease, wherein the subject data comprises an indication of whether a biomarker associated with the disease is present; obtain a plurality of candidate best fit models based on the subject data by: i) providing a predefined estimate of a number of subgroups; ii) generating a set of models based on the subject data, each model of the set comprising the same predefined estimate of the number of subgroups; iii) selecting a candidate best fit model from the set of models; and iv) repeating (i)-(iii) at least once using a different predefined estimate of the number of subgroups to obtain a plurality of candidate best fit models; select a best fit model from the plurality of candidate best fit models based on a fit statistic; and determine a number of subgroups for the disease and an associated genomic profile for each subgroup based on the best fit model and subject data.

In some embodiments, the system further comprises instructions that, when executed by the one or more processors, cause the system to determine to which disease subgroup an individual subject belongs based on the individual subject's data and the best fit model. In some embodiments, the system further comprises instructions that, when executed by the one or more processors, cause the system to provide a treatment recommendation or outcome prediction for the individual subject based on the disease subgroup to which the individual subject belongs. In some embodiments, the system further comprises instructions that, when executed by the one or more processors, cause the system to identify a subgroup of subjects for participation in a clinical study based on a disease subgroup to which the subgroup of subjects belong. In some embodiments, the system further comprises instructions that, when executed by the one or more processors, cause the system to identify one or more driver mutations for a disease subgroup based on the associated genomic profile for the subgroup. In some embodiments, the plurality of subjects have been diagnosed to have cancer.

Disclosed herein are non-transitory computer-readable storage media storing one or more programs, the one or more programs comprising instructions, which when executed by one or more processors of a system, cause the system to: receive subject data for a plurality of subjects diagnosed with a disease, wherein the subject data comprises an indication of whether a biomarker associated with the disease is present; obtain a plurality of candidate best fit models based on the subject data by: i) providing a predefined estimate of a number of subgroups; ii) generating a set of models based on the subject data, each model of the set comprising the same predefined estimate of the number of subgroups; iii) selecting a candidate best fit model from the set of models; and iv) repeating (i)-(iii) at least once using a different predefined estimate of the number of subgroups to obtain a plurality of candidate best fit models; select a best fit model from the plurality of candidate best fit models based on a fit statistic; and determine a number of subgroups for the disease and an associated genomic profile for each subgroup based on the best fit model and the subject data.

In some embodiments, the non-transitory computer-readable storage medium further comprises instructions that, when executed by the one or more processors, cause the system to determine to which disease subgroup an individual subject belongs based on the individual subject's data and the best fit model. In some embodiments, the non-transitory computer-readable storage medium further comprises instructions that, when executed by the one or more processors, cause the system to provide a treatment recommendation or outcome prediction for the individual subject based on the disease subgroup to which the individual subject belongs. In some embodiments, the non-transitory computer-readable storage medium further comprises instructions that, when executed by the one or more processors, cause the system to identify a subgroup of subjects for participation in a clinical study based on a disease subgroup to which the subgroup of subjects belong. In some embodiments, the non-transitory computer-readable storage medium further comprises instructions that, when executed by the one or more processors, cause the system to identify one or more driver mutations for a disease subgroup based on the associated genomic profile for the subgroup. In some embodiments, the plurality of subjects have been diagnosed to have cancer.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A provides a non-limiting example of a process flowchart for identifying disease subgroups based on patient data for a plurality of patients diagnosed with a disease according to one embodiment of the present disclosure.

FIG. 1B provides an exemplary schematic illustration of a process for generating sets of models for different predetermined estimates of the number of disease subgroups represented in a dataset for a patient cohort, thereby generating a plurality of candidate best fit models, and for selecting a best fit model from the plurality of candidate best fit models according to one embodiment of the present disclosure.

FIG. 2 depicts an exemplary computing device or system in accordance with one embodiment of the present disclosure.

FIG. 3 depicts an exemplary computer system or computer network in accordance with one embodiment of the present disclosure.

FIG. 4A provides a non-limiting example of genomic landscape data for a cohort of young adult glioblastoma (yaGBM) patients.

FIG. 4B provides a non-limiting example of genomic landscape data for a cohort of young adult glioblastoma with primitive neuroectodermal tumor feature (yaGBM-PNET) patients.

FIG. 4C provides a non-limiting example of genomic landscape data for a cohort of young adult glioblastoma with oligodendroglial tumor feature (yaGBM-O) patients.

FIG. 5A provides a non-limiting example of mutation rate data for the 25 most frequently altered genes in yaGBM patients compared to that for classic adult GBM patients using a series of Fisher Exact tests with Holm-Bonferroni Correction.

FIG. 5B provides a non-limiting example of mutation rate data for the 25 most frequently altered genes in yaGBM patients compared to that for pediatric GBM patients using a series of Fisher Exact tests with Holm-Bonferroni Correction.

FIG. 5C provides a non-limiting example of mutation rate data for the 25 most frequently altered genes in yaGBM patients with IDH1/2 mutations compared to that for yaGBM patients without IDH1/2 mutations.

FIG. 6A provides a non-limiting example of genomic landscape data for a cohort of IDH wild-type yaGBM patients.

FIG. 6B provides a non-limiting example of genomic landscape data for a cohort of IDH mutant yaGBM patients.

FIG. 7A provides a non-limiting example of genomic landscape data for cohort of H3F3A mutant yaGBM patients.

FIG. 7B provides a non-limiting example of genomic landscape data for a cohort of BRAF mutant yaGBM patients.

FIG. 8A provides a non-limiting example of a plot of tumor mutational burden (TMB) distribution in yaGBM patients.

FIG. 8B provides a non-limiting example of a plot of tumor mutational burden (TMB) score versus the mutational signature in yaGMB patients.

FIG. 9A provides a non-limiting example of genomic landscape data for latent molecular classes (disease subgroups) in yaGBM patients.

FIG. 9B provides a non-limiting example of a chart illustrating ten distinct molecular classes (disease subgroups) identified for yaGBM patients.

### DETAILED DESCRIPTION

Methods and systems are described for classification of disease entities to identify disease subgroups based on genomic data (alone, or in combination with other clinical data) for a cohort of patients that have been diagnosed with the disease, and to identify genomic profiles and driver mutations associated with each disease subgroup. The methods may comprise, for example, processing data for a plurality of patients diagnosed with a disease, where the patient data (e.g., patient genomic data and/or patient characteristics such as age, gender, height, weight, etc.) comprises an indication of whether a biomarker associated with the disease is present in each patient of the plurality; generating a plurality of candidate best fit models based on the subject data by iteratively estimating the number of disease subgroups to be identified, generating a set of models based on the current estimate of the number of disease subgroups to be identified, selecting a candidate best fit model from the set of models, and repeating these steps using a different estimate of the number of disease subgroups; selecting a best fit model from the plurality of candidate best fit models based on a fit statistic; and applying the best fit model to the patient data to identify a number of subgroups for the disease and an associated genomic profile for each subgroup.

In some embodiments, the disclosed methods may rely on statistical (e.g., latent class analysis) and/or probabilistic (e.g., mixture modeling) techniques to identify patterns of association between observable variables for a population (e.g., the presence or absence of specific genomic alterations and/or other clinical characteristics in a patient cohort) and a set of latent variables (e.g., variables that are not directly observed but are inferred through a statistical model based on other variables that are observed) that define a set of latent classes (i.e., disease subgroups) that make up the population. Latent class analysis may be performed with the objective of identifying latent classes (disease subgroups) for which the observable variables (e.g., the presence or absence of specific genomic alterations, etc.) are “conditionally independent”, i.e., there is no longer an association between one observable variable and another observable variable except in the context of membership in one of the identified latent classes. Similarly, mixture modeling may be performed to make statistical inferences about the properties of sub-populations (disease subgroups) within a population of, e.g., patients diagnosed with the disease, based on data for observable variables (e.g., the presence or absence of specific genomic alterations, etc.) for the patient cohort without prior knowledge of the number or defining characteristics of the sub-populations that are present.

The disclosed methods and systems provide advantages over previous approaches in terms of the ability to successfully categorize patients according to disease subgroups that comprise distinctive driver mutations. Furthermore, the data modeling may be more instructive for describing actionable and/or prognostic features for different disease subgroups. The disclosed methods for categorizing patients according to disease subgroups and identifying driver alterations in different disease subgroups has the potential to provide greater insight into patient disease characteristics and treatment options, facilitate treatment decisions by healthcare providers, improve treatment outcome predictions, and to facilitate the design of clinical trials and development of new therapeutics. In some embodiments, the disclosed methods and systems may also enable the use of smaller bait sets for complete genomic profiling assays, reduce the costs associated with sequencing nucleic acid samples derived from patients, and reduce the amount of patient sequencing data that needs to be processed and stored.

In some instances, for example, methods are described that comprise receiving, at one or more processors, patient data for a plurality of patients diagnosed with the disease, wherein the patient data comprises an indication of whether a biomarker associated with the disease is present; obtaining, using the one or more processors, a plurality of candidate best fit models based on the patient data by: i) providing a predefined number of subgroups; ii) generating a set of models based on the patient data, each model of the set comprising the same predefined number of subgroups; iii) selecting a candidate best fit model from the set of models; and iv) repeating (i)-(iii) at least once using a different predefined number of subgroups to obtain a plurality of candidate best fit models; selecting a best fit model from the plurality of candidate best fit models based on a fit statistic; and determining, using the one or more processors, a number of subgroups for the disease and an associated genomic profile for each subgroup based on the best fit model and the patient data.

In some instances, the method further comprises determining to which disease subgroup an individual patient belongs based on the individual patient's data and the best fit model. In some instances, the method further comprises providing a treatment recommendation or outcome prediction for the individual patient based on the disease subgroup to which the individual patient belongs. In some instances, the method further comprises identifying a subgroup of patients for participation in a clinical study based on a disease subgroup to which the subgroup of patients belong. In some instances, the method further comprises identifying one or more driver mutations for a disease subgroup based on the associated genomic profile for the subgroup.

In some instances, the plurality of candidate best fit models comprise non-probabilistic models. In some instances, the plurality of candidate best fit models comprise probabilistic models. In some instances, the models of the set of models comprising the same predefined number of subgroups are generated using a clustering or mixture modeling technique.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Methods for Classification of Disease Entities:

Methods are described for classification of disease entities to identify disease subgroups based on genomic data (alone, or in combination with other patient characteristics or clinical data) for a cohort of patients that have been diagnosed with the disease, and to identify genomic profiles and driver mutations associated with each disease subgroup. Cancer, for example, is driven by onco-driving mutations (e.g., the KRAS oncogene in colorectal cancer, BRAF V600E mutations in multiple disease areas, etc.). It is thus known that important molecular drivers of cancer exist, and that they vary by tumor type, but the process of extracting salient features from each tumor type is difficult. The methods described herein may encompass: (i) processing patient data (e.g., genomic data and/or other patient characteristics or clinical data) for a cohort of patients that have been diagnosed as having a disease based on, e.g., conventional histopathology reports, (ii) using the patient data to train and evaluate a plurality of candidate models (e.g., statistical models or machine learning models) to identify disease subgroups based on a set of initial guesses for how many disease subgroups are present, (iii) selecting a best fit model from the candidate models based on an overall data fit statistic and how well it separates each disease subgroup present in the data, (iv) providing computed values of model parameters that characterize each disease subgroup (e.g., a mean value for a distribution of genomic alterations that characterize the disease subgroup and a covariance term for each of two or more genomic alterations with the disease subgroup), and (v) predicting the expected prevalence for each disease subgroup (e.g., the relative proportion of each disease subgroup within the patient cohort).

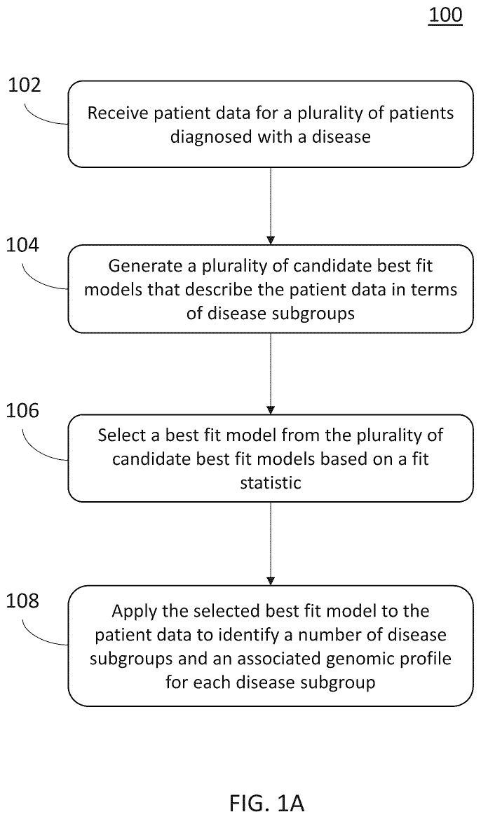

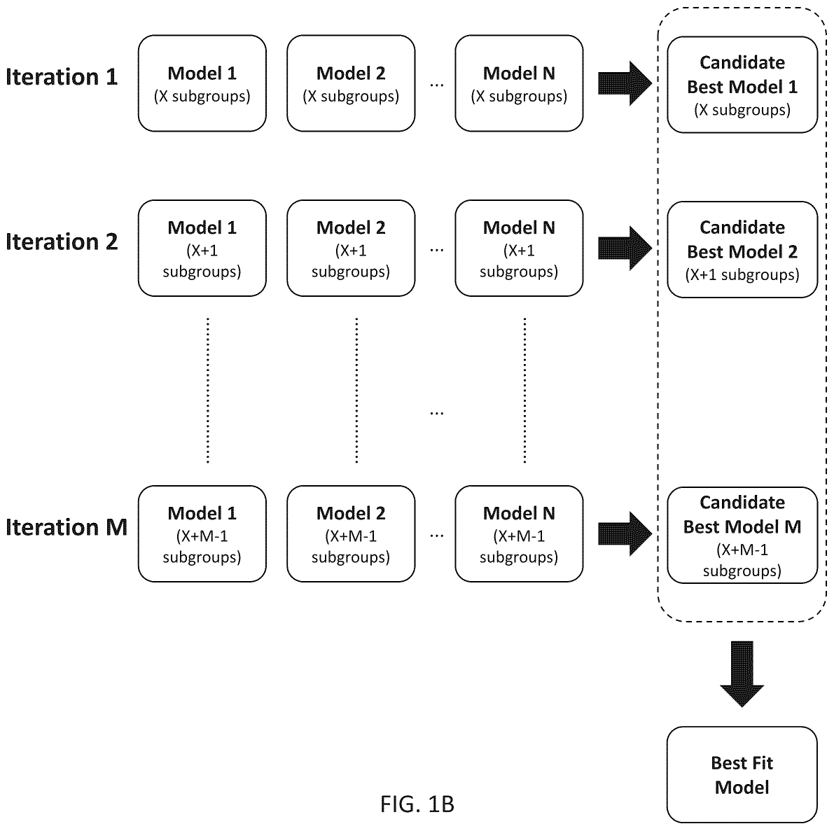

FIG. 1A provides a non-limiting example of a flowchart for a process 100 for identifying disease subgroups based on patient data (or subject data) for a plurality of patients (or subjects or individuals) that have been diagnosed with a disease. Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1A, patient data is received for a plurality of patients that have been diagnosed with a disease. The patient data may be received, e.g., by one or more processors of a system configured to perform the methods described herein. In some instances, the patient data may comprise genomic data (or genomic profile data) for each patient of the plurality of patients (e.g., genomic data (or genomic profile data) generated by sequencing nucleic acid molecules extracted from a patient sample as described elsewhere herein).

In some instances, the genomic data (or genomic profile data) may comprise an indication of whether a biomarker associated with the disease is present in a given patient's genome. In some instances, the biomarker associated with the disease may comprise one or more genomic alterations, e.g., mutations in one or more genes and/or one or more chromosomes. In some instances, a mutation (or alteration) may comprise, e.g., a point mutation, insertion, deletion, duplication, copy number variation (CNV), rearrangement, inversion, substitution, translocation, fusion, or any combination thereof, in one or more genes and/or one or more chromosomes (or portions thereof).

In some instances, the biomarker may comprise one or more genomic alterations, e.g., mutations, in at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, or more than 500 genes, or portions thereof. In some instances, the biomarker may comprise genomic alterations in at least 5 genes but in less than 100 genes, in at least 10 genes but less than 90 genes, in at least 15 genes but less than 80 genes, in at least 20 genes but less than 70 genes, or in at least 25 genes but less than 60 genes. In some instances, the biomarker may comprise one or more genomic alterations, e.g., mutations, in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46 chromosomes (e.g., human chromosomes).

In some instances, the biomarker associated with the disease may further comprise, for example, variants in one or more gene loci that are not typically considered pathogenic, immunohistochemical staining results for one or more biomarker proteins (e.g., where a predetermined threshold is applied to determine clinical relevance), amplifications or deletions of chromosome arms, entire chromosomes and other cytogenetic events, or any other lab marker identified by a clinical test (e.g., albumin blood test, beta-hCG, CA19-9, HE4, or the like).

In some instances, the genomic data (or genomic profile data) may comprise a binary indication of whether a pathogenic mutation is present in each gene of a specified panel of genes. In some instances, the genomic data (or genomic profile data) may be received in the form of a matrix comprising M rows×N columns, where each of the M rows comprises data for an occurrence of a genetic mutation in a given gene of the specified panel of genes in an individual patient, and each of the N columns comprises genetic mutation data across the specified panel of genes for an individual patient of the plurality of patients.

In some instances, the patient data may further comprise clinical data for all or a portion of the patients of the plurality, e.g., data regarding patient sex, patient age, patient gender, patient height, patient weight, patient clinical history, patient sample type, or any combination thereof.

In some instances, the disease with which the patients have been diagnosed may comprise a monogenic disorder (including two or more disease subgroups in the monogenic disorder), a polygenic disorder, or a multifactorial disorder. In some instances, the disease may comprise an inherited disease. In some instances, the disease may comprise an acquired disease. In some instances, the disease may comprise a multifactorial inherited or acquired disease, e.g., diseases caused by genomic alterations in one or more genes and/or chromosomes in combination with one or more additional factors (such as nutrition, lifestyle, alcohol or tobacco use, exposure to pollutants, etc.).

Examples of monogenic hereditary diseases or conditions include, but are not limited to, al-antitrypsin deficiency, cystic fibrosis, familial hypercholesterolemia, Huntington's disease, sickle cell disease, Tay-Sachs disease, phenylketonuria, and polycystic kidney disease.

Examples of polygenic hereditary diseases or conditions include, but are not limited to, cleft palate, congenital heart defects, congenital hip dysplasia, neural tube defects, pyloric stenosis, and type 1 diabetes.

Examples of monogenic or polygenic acquired diseases or conditions include, but are not limited to, many forms of cancer, Ehlers-Danlos syndrome, Lynch syndrome, or the like.

Examples of multifactorial inherited or acquired diseases or conditions include, but are not limited to, arthritis, autoimmune disease, breast cancer, coronary heart disease, hypertension, lung cancer, ovarian cancer, prostate cancer, skin cancer, other cancers described elsewhere herein, and type 2 diabetes.

At step 104 in FIG. 1A, a plurality of candidate best fit models are generated that describe the patient data for the plurality of patients in terms of disease subgroups. As noted above, the disclosed methods rely on statistical (e.g., latent class analysis) and/or probabilistic (e.g., mixture modeling) techniques to identify patterns of association between observable variables for a population (e.g., the presence or absence of specific genomic alterations and/or other clinical characteristics in a patient cohort) and a set of latent variables that define a set of latent classes or sub-populations (i.e., disease subgroups) that make up the population.

In some instances, for example, the plurality of candidate best fit models may be generated by: (i) providing a predefined estimate of the number of disease subgroups present in the population; (ii) generating a set of non-probabilistic or probabilistic models (e.g., latent class models and/or mixture models) based on the patient data, where each model of the set comprises the same predefined estimate of the number of disease subgroups but differs in, e.g., the set of initialization parameters used to generate a given model of the set; (iii) selecting a candidate best fit model from the set of models; and (iv) repeating steps (i)-(iii) at least once using a different predefined number of subgroups to obtain a plurality of candidate best fit models.

FIG. 1B provides a schematic illustration of the process for generating the plurality of candidate best fit models described above and at step 104 in FIG. 1A. The patient data received in step 102 in FIG. 1A may be used to generate a first model based on a first estimate of the number of disease subgroups (e.g., X subgroups) represented by the patient data using a first set of initialization parameters. A second model may be then generated based on the first estimate of the number of disease subgroups (e.g., X subgroups) represented by the patient data using a second, different set of initialization parameters. These steps may be repeated for up to N times, as illustrated in FIG. 1B, so that a set of N models are generated based on the first estimate of the number of disease subgroups (e.g., X subgroups) using N different sets of initialization parameters. Once the set of N models has been generated, a candidate best fit model may be selected from the set, e.g., by selecting the model that optimizes an objective function, as described elsewhere herein. The entire process may then be repeated for up to M iterations, as illustrated in FIG. 1B, using different estimates of the number of disease subgroups represented in the patient data (e.g., X+1, X+2, . . . . X+M−1 subgroups) for each iteration to generate a set of M candidate best fit models. A best fit model may then be selected from the set of M candidate best fit models based on, e.g., a fit statistic, as described below and indicated at step 106 in FIG. 1A.

In some instances, the predefined estimate of the number of disease subgroups present in the population may be based on, e.g., the number of subgroups identified in the patient data using a variational Bayesian method (e.g., any of a family of a family of techniques used in statistical modeling to infer the conditional distribution of a set of observed data and unknown parameters over a set of latent variables) or agglomerative clustering technique (e.g., an unsupervised machine learning technique that divides a population data set into several clusters such that data points in the same cluster are similar and data points in different clusters are dissimilar).

In some instances, the predefined estimate of the number of subgroups (e.g., disease subgroups) may range from 1 to 100. In some instances, the predefined estimate of the number of subgroups (e.g., disease subgroups) may range from 1 to 50. In some instances, the predefined estimate of the number of subgroups (e.g., disease subgroups) may range from 1 to 20. In some instances, the predefined estimate of the number of subgroups (e.g., disease subgroups) may be 2, 3, 4, 5, 6, 7, 8, 9, 10, 20, 30, 40, 50, 60, 70, 80, 90, 100, or any number within this range.

In some instances, the set of non-probabilistic or probabilistic models comprise non-probabilistic models (e.g., latent class models, hard clustering techniques, etc.). In some instances, the set of non-probabilistic or probabilistic models comprise probabilistic models (e.g., soft clustering techniques, mixture models, etc.).

Examples of suitable clustering techniques include, but are not limited to, k-means clustering, mean-shift clustering, density-based spatial clustering of applications with noise (DBSCAN) techniques, agglomerative hierarchical clustering, random forest techniques, or any combination thereof.

In some instances, the clustering technique may comprise, for example, a k-means clustering technique, and the output of the model comprises, for each disease subgroup (i.e., each cluster), a centroid, an intra-cluster distance, an inertia value, an inter-cluster distance for each pair of clusters, a Dunn index, an expected prevalence for each cluster, or any combination thereof.

Examples of suitable mixture models include, but are not limited to, Gaussian mixture models, multivariate Gaussian mixture models, categorical mixture models, variational Bayes mixture models, or any combination thereof.

In some instances, the mixture modeling technique may comprise, for example, a Gaussian mixture modeling technique, and the output of the model comprises, for each subgroup, a mean, μ, a covariance, Σ, and a mixing probability, π, an expected prevalence, or any combination thereof. In some instances, optimal values for one or more model parameters may be determined by determining a maximum likelihood estimate.

In some instances, each model in a set of models that comprises the same predefined estimate of the number of disease subgroups differs in, e.g., a set of initialization parameters used to generate the model. For example, in some instances the different sets of initialization parameters comprise sets of randomly assigned initialization parameters. Examples of initialization parameters include, but are not limited to, the type of model used, a tolerance on log-likelihood function in each epoch of an iterative modeling technique, the maximum number of epochs allowed for iterative modeling, instructions for calculating co-variance, initial guesses or values for the midpoints for each Gaussian distribution in the case that a Gaussian mixture model technique is used, and the like.

In some instances, the different sets of initialization parameters comprise initialization parameters generated using a k-nearest neighbor (k-NN) method. k-NN is a supervised machine learning technique used for classification or regression, where the input comprises the number of nearest neighbors, k, in a training data set (i.e., a set of known, labeled data) used to evaluate or classify an unknown test data point based on a specified distance metric (e.g., a Euclidian distance for continuous variables, or a Hamming distance for discrete variables), and the output comprises a class designation (for classification; based e.g., on the mode of the label values for the k nearest neighbors) or property value (for regression; based, e.g., on the mean of the label values for the k nearest neighbors) for the initially unknown test data point. In some instances, a-kNN model may be used to provide, e.g., the initial estimates for the midpoints for each Gaussian distribution in a Gaussian mixture model based on the patient data for the plurality of patients and the predetermined estimate of the number of disease subgroups. A set of models for a given predetermined estimate of the number of disease subgroups may then be generated using initialization conditions, e.g., initial estimates for the midpoints for each Gaussian distribution in a Gaussian mixture model, determined for values of k=1, 2, 3, 4, 5, 6, 7, 8, 9, 10, etc.

In some instances, the method further comprises specifying a set of one or more operational parameters for generating the set of models comprising the same predefined number of subgroups, where the specified operational parameters comprise a maximum number of epochs to be performed, a tolerance on log-likelihood function for each epoch, a metric for calculation of covariance for each epoch, a metric (e.g., alpha) that controls a degree to which a new subgroup will form, or any combination thereof.

In some instances, the set of models generated for each predefined estimate of the number of disease subgroups comprises from 2 to 20 models. In some instances, the set of models may comprise 2, 3, 4, 5, 6, 7, 8, 9, 10, 11, 12, 13, 14, 15, 16, 17, 18, 19, 20, or more than 20 models.

In some instances, selecting a candidate best fit model from the set of models (i.e., the set of models generated for the same predefined estimate of the number of disease subgroups) comprises selecting the model that optimizes an objective function (e.g., a loss function, cost function, or a likelihood function). For example, in some instances, selecting a candidate best fit model from the set of models comprises selecting the model that optimizes a log-likelihood function. In some instances, the selected candidate best fit model is the model which has a maximum log-likelihood value.

As noted above, in some instances, a plurality of candidate best fit models may be generated by repeating the steps of (i) providing a predefined estimate of the number of disease subgroups present in the population; (ii) generating a set of non-probabilistic or probabilistic models (e.g., latent class models and/or mixture models) based on the patient data, where each model of the set comprises the same predefined estimate of the number of disease subgroups but differs in, e.g., the set of initialization parameters used to generate a given model of the set; and (iii) selecting a candidate best fit model from the set of models, at least once using a different predefined number of subgroups for each iteration. In some instances, steps (i)-(iii) may be repeated at least 1 time, at least 2 times, at least 3 times, at least 4 times, at least 5 times, at least 6 times, at least 7 times, at least 8 times, at least 9 times, or at least 10 times, using a different predefined estimate of the number of subgroups each time.

At step 106 in FIG. 1A, a best fit model is selected from the plurality of candidate best fit models generated at step 104A based on, for example, a fit statistic that describes goodness of fit relative to the number of parameters (e.g., the number of disease subgroups) that the model comprises. Examples of suitable fit statistics include, but are not limited to, an Akaike Information Criterion (AIC), a Bayesian Information Criterion (BIC), a Calinski-Harabasz (CH) score, or any combination thereof. In some instances, the best fit model is the model from the plurality of candidate best fit models that maximizes the fit statistic. In some instances, the first local maxima in a plot of fit statistic (e.g., CH score) versus number of subgroups was used to select the best fit model.

The Akaike Information Criterion (AIC) is given by: AIC=2k−2 ln (L), where k is the number of estimated parameters in the model and L is the maximum value of the likelihood function for the model. The AIC provides an estimate of the relative quality of a given model in a set of statistical models for a given set of data, and thus, provides a means for selecting a best fit model from the set.

The Bayesian Information Criterion (BIC) is given by: BIC=k ln (n)−2 ln (L) where k is the number of parameters estimated by the model, n is the number of data points in the set of observed data, x, and L is the maximum value of the likelihood function of the model M, i.e., L=p(x|□, M), where □ are the set of parameter values that maximize the likelihood function.

The Calinski-Harabasz (CH) score (also known as the Variance Ratio Criterion) can be used to evaluate models when ground truth labels are not known, and the validation of how well the grouping (or clustering) of the data has been performed is based on quantities and features inherent to the dataset. The CH score is given by the ratio of the sum of between-group variance (or the sum of between cluster dispersion) and within-group variance (or inter-cluster dispersion) for all groups (or clusters; e.g., disease subgroups), where the higher the score the better the performance of the model:

$${S}_{C H} = \frac{( N - K ) B}{( K - 1 ) {\sum }_{k = 1}^{K} {W}_{k}}$$

where N is the number of observations or data points, K is the number of groups (e.g., disease subgroups), B is the between-group variance, and Wₖ is the within-group variance:

$$B = {\sum }_{k = 1}^{K} \vert {I}_{k} \vert ‖ {μ}_{k} - μ ‖$$

$${W}_{k} = {\sum }_{i ∈ {I}_{k}} ‖ {x}^{i} - {μ}_{k} ‖$$

where Iₖ is the set of data points belonging to group k, μₖ is the mid-point of group k, and u is the mid-point of the entire data set.

At step 108 in FIG. 1A, the selected best fit model is applied to the patient data to identify a number of disease subgroups and an associated genomic profile for each disease subgroup.

In some instances, the number of subgroups (e.g., disease subgroups) identified may range from 1 to 100. In some instances, the number of subgroups (e.g., disease subgroups) identified may range from 1 to 50. In some instances, the number of subgroups (e.g., disease subgroups) identified may range from 1 to 20. In some instances, the number of subgroups (e.g., disease subgroups) identified may be 2, 3, 4, 5, 6, 7, 8, 9, 10, 20, 30, 40, 50, 60, 70, 80, 90, 100, or any number within this range.

In some instances, the disclosed methods may further comprise determining to which disease subgroup an individual patient belongs based on the individual patient's data and the best fit model.

In some instances, the disclosed methods may further comprise providing a treatment recommendation or outcome prediction for the individual patient based on the disease subgroup to which the individual patient belongs.

In some instances, the disclosed methods may further comprise identifying a subgroup of patients for participation in a clinical study based on a disease subgroup to which the subgroup of patients belong.

In some instances, the disclosed methods may further comprise identifying one or more driver mutations for a disease subgroup based on the associated genomic profile for the subgroup.

In some instances, the disclosed methods may further comprise modifying a specified panel of genes used for genomic profiling of the disease based on the identified number of subgroups for the disease and the associated genomic profile for each subgroup. In some instances, modification of the specified panel of genes may comprise modifying a bait set used to generate genomic profile data. In some instances, the specified panel of genes may comprise between 5 and 100 genes for which there are known pathogenic or likely pathogenic mutations. In some instances, the specified panel of genes may comprise between 10 and 100 genes for which there are known pathogenic or likely pathogenic mutations. In some instances, the specified panel of genes may comprise at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 genes (or any number of genes within this range) for which there are known pathogenic or likely pathogenic mutations.

In some instances, the specified panel of genes may comprise the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRβ, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for identifying disease subgroups may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for identifying disease subgroups may be used to select a subject (e.g., a patient) for a clinical trial based on the disease subgroup to which the subject belongs. In some instances, patient selection for clinical trials based on, e.g., identification of a disease subgroup to which the patient belongs, may accelerate the development of targeted therapies and improve the healthcare outcomes for treatment decisions.

In some instances, the disclosed methods for identifying disease subgroups may be used to select an appropriate therapy or treatment (e.g., an anti-cancer therapy or anti-cancer treatment) for a subject based on the disease subgroup to which the subject belongs. In some instances, for example, the anti-cancer therapy or treatment may comprise use of a poly (ADP-ribosc) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/ Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denilcukin diftitox (Ontak), denosumab (Xgeva), dinutuximab (Unituxin), dostarlimab-gxly (Jemperli), durvalumab (Imfinzi), duvelisib (Copiktra), clotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligco), moxctumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumctinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for identifying disease subgroups may be used in treating a disease (e.g., a cancer) in a subject. For example, in response to determining a disease subgroup to which a subject belongs using any of the methods disclosed herein, an effective amount of an anti-cancer therapy or anti-cancer treatment may be administered to the subject.

In some instances, the disclosed methods for identifying disease subgroups may be used for monitoring disease progression or recurrence (e.g., cancer or tumor progression or recurrence) in a subject. For example, in some instances, the methods may be used to determine a disease subgroup to which the subject belongs in a first sample obtained from the subject at a first time point, and used to determine a disease subgroup to which the subject belongs in a second sample obtained from the subject at a second time point, where comparison of the first determination of disease subgroup and the second determination of disease subgroup allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of the disease subgroup to which the subject belongs.

In some instances, the disease subgroup to which a subject belongs, as determined using the disclosed methods, may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for identifying disease subgroups may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for identifying disease subgroups as part of a genomic profiling process (or inclusion of the output from the disclosed methods for identifying a disease subgroup to which a subject belongs as part of the genomic profile of the subject) can improve the validity of, e.g., disease detection calls and treatment decisions, made on the basis of the genomic profile.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo (dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic cosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22): 4436-4443; Specht, et al., (2001) Am J Pathol. 158 (2): 419-429; the Ambion RecoverAllTM Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or nonspecific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4 (11): 903-5; Hodges, E. et al. (2007) Nat. Genet. 39 (12): 1522-7; and Okou, D. T. et al. (2007) Nat. Methods 4 (11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 103, 104, 105 or more than 105 molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (c) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (scc, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. COT in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26 (5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85 (6): 847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜ le-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20 (9): 1297-303; Ye, K., et al., Bioinformatics, 2009; 25 (21): 2865-71; Lunter, G., and Goodson, M., Genome Res. 2011; 21 (6): 936-9; and Li, H., et al. (2009), Bioinformatics 25 (16): 2078-9.

Methods for generating indel calls and individual-level genotype likelihoods include, e.g., the Dindel algorithm (Albers, C. A., et al., Genome Res. 2011; 21 (6): 961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21 (6): 952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26 (6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for identifying disease subgroups for a given disease, and for identifying a disease subgroup to which a subject belongs based on analysis of a sample from a subject. The systems may comprise, e.g., one or more processors, and a memory unit communicatively coupled to the one or more processors and configured to store instructions that, when executed by the one or more processors, cause the system to: receive patient data for a plurality of patients diagnosed with the disease, wherein the patient data comprises an indication of whether a biomarker associated with the disease is present; obtain a plurality of candidate best fit models based on the patient data by: (i) providing a predefined estimate of a number of subgroups; (ii) generating a set of models based on the patient data, each model of the set comprising the same predefined estimate of the number of subgroups; (iii) selecting a candidate best fit model from the set of models; and (iv) repeating (i)-(iii) at least once using a different predefined estimate of the number of subgroups to obtain a plurality of candidate best fit models; select a best fit model from the plurality of candidate best fit models based on a fit statistic; and determine a number of subgroups for the disease and an associated genomic profile for each subgroup based on the best fit model and the patient data.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for identifying disease subgroups for a given disease, and for identifying a disease subgroup to which a subject belongs based on analysis of a variety of samples as described herein (e.g., a tissue sample, biopsy sample, hematological sample, or liquid biopsy sample derived from the subject).

In some instances, the plurality of gene loci for which sequencing data is processed to determine the number of disease subgroups that exist for a given disease and/or to determine in which disease subgroup an individual subject belongs may comprise at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 genes (or any number of genes within this range).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a disease subgroup to which a subject (e.g., a patient) belongs may be used to select, initiate, adjust, or terminate a treatment for cancer in the subject from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

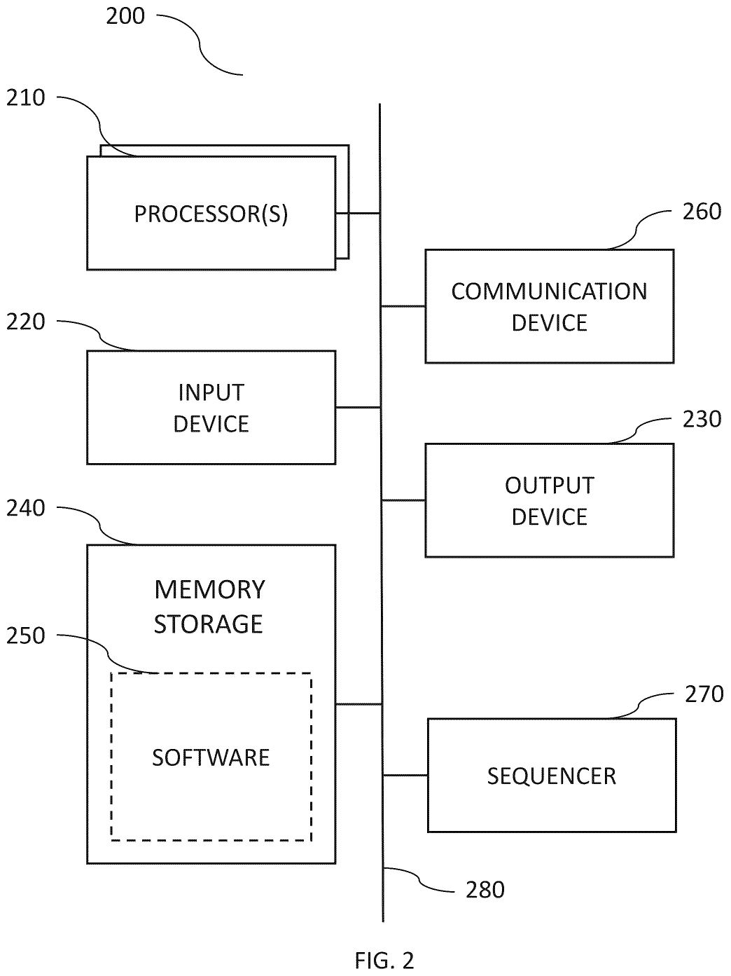

FIG. 2 illustrates an example of a computing device or system in accordance with one embodiment. Device 200 can be a host computer connected to a network. Device 200 can be a client computer or a server. As shown in FIG. 2, device 200 can be any suitable type of microprocessor-based device, such as a personal computer, workstation, server or handheld computing device (portable electronic device) such as a phone or tablet. The device can include, for example, one or more processor(s) 210, input devices 220, output devices 230, memory or storage devices 240, communication devices 260, and nucleic acid sequencers 270. Software 250 residing in memory or storage device 240 may comprise, e.g., an operating system as well as software for executing the methods described herein. Input device 220 and output device 230 can generally correspond to those described herein, and can either be connectable or integrated with the computer.

Input device 220 can be any suitable device that provides input, such as a touch screen, keyboard or keypad, mouse, or voice-recognition device. Output device 230 can be any suitable device that provides output, such as a touch screen, haptics device, or speaker.

Storage 240 can be any suitable device that provides storage (e.g., an electrical, magnetic or optical memory including a RAM (volatile and non-volatile), cache, hard drive, or removable storage disk). Communication device 260 can include any suitable device capable of transmitting and receiving signals over a network, such as a network interface chip or device. The components of the computer can be connected in any suitable manner, such as via a wired media (e.g., a physical system bus 280, Ethernet connection, or any other wire transfer technology) or wirelessly (e.g., Bluetooth®, Wi-Fi®, or any other wireless technology).

Software module 250, which can be stored as executable instructions in storage 240 and executed by processor(s) 210, can include, for example, an operating system and/or the processes that embody the functionality of the methods of the present disclosure (e.g., as embodied in the devices as described herein).

Software module 250 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 240, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

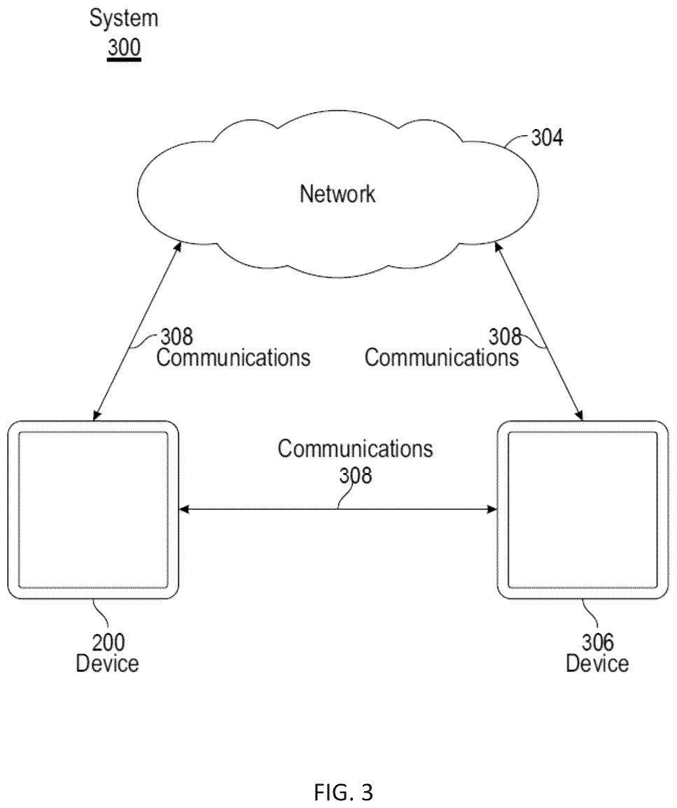

Device 200 may be connected to a network (e.g., network 304, as shown in FIG. 3 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 200 can be implemented using any operating system, e.g., an operating system suitable for operating on the network. Software module 250 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 210.

Device 200 can further include a sequencer 270, which can be any suitable nucleic acid sequencing instrument.

FIG. 3 illustrates an example of a computing system in accordance with one embodiment. In system 300, device 200 (e.g., as described above and illustrated in FIG. 2) is connected to network 304, which is also connected to device 306. In some embodiments, device 306 is a sequencer. Exemplary sequencers can include, without limitation, Roche/454's Genome Sequencer (GS) FLX System, Illumina/Solexa's Genome Analyzer (GA), Illumina's HiSeq® 2500, HiScq® 3000, HiScq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 200 and 306 may communicate, e.g., using suitable communication interfaces via network 304, such as a Local Area Network (LAN), Virtual Private Network (VPN), or the Internet. In some embodiments, network 304 can be, for example, the Internet, an intranet, a virtual private network, a cloud network, a wired network, or a wireless network. Devices 200 and 306 may communicate, in part or in whole, via wireless or hardwired communications, such as Ethernet, IEEE 802.11b wireless, or the like. Additionally, devices 200 and 306 may communicate, e.g., using suitable communication interfaces, via a second network, such as a mobile/cellular network. Communication between devices 200 and 306 may further include or communicate with various servers such as a mail server, mobile server, media server, telephone server, and the like. In some embodiments, Devices 200 and 306 can communicate directly (instead of, or in addition to, communicating via network 304), e.g., via wireless or hardwired communications, such as Ethernet, IEEE 802.11b wireless, or the like. In some embodiments, devices 200 and 306 communicate via communications 308, which can be a direct connection or can occur via a network (e.g., network 304).

One or all of devices 200 and 306 generally include logic (e.g., http web server logic) or are programmed to format data, accessed from local or remote databases or other sources of data and content, for providing and/or receiving information via network 304 according to various examples described herein.

### EXAMPLES

### Example 1 Genomic Landscape of Young Adult Glioblastomas

This section provides a non-limiting example of the application of the disclosed methods to the analysis of glioblastomas in young adults. Glioblastomas (GBM) are primary malignant brain tumors that develop across all age groups, including children and adults. Standard of care treatment includes maximal surgical resection followed by chemotherapy (e.g. temozolomide) and radiation therapy; however, median overall survival (OS) remains poor at 14.6 months.

Integration of next-generation sequencing (NGS) into clinical workflows enables clinicians to identify genomic alterations with targeted therapies and/or prognostic value for many tumor types. While brain tumors have traditionally been classified on a histopathological basis, the World Health Organization (WHO)'s system for classification of tumors of the central nervous system has recently integrated genomic biomarkers into their diagnostic indications. Among adult WHO Grade 2/3 brain tumors, tumors of astrocytic lineage are associated with IDH1/2, TP53, and ATRX mutations, while oligodendroglial lineage tumors are associated with mutations in CIC, FUBP1, and 1p/19q co-deletion.

In this study, the genomic landscape for a cohort of 661 young adult GBMs (yaGBM), was analyzed using complete genomic profiling (CGP) to define unique molecular subgroups. The analysis explored variants associated with evidence of pathogenic function, common copy number variants (CNVs), and several novel fusion events not described in literature. The analysis further explored tumor mutational burden (TMB) mutational signatures, anatomic location, and tumor recurrence. Using data collected from CGP, unsupervised machine learning techniques were leveraged to identify ten genomic classes of yaGBMs. The current descriptions in the literature were used to link these classes with clinically- and prognostically-valuable descriptions where possible.

### Methods

Comprehensive Genomic Profiling: Comprehensive genomic profiling (CGP) was performed in a College of American Pathologists (CAP)-accredited, Clinical Laboratory Improvement Amendments (CLIA)-certified, New York State regulated reference laboratory. All samples underwent central histopathologic review by a board-certified neuropathologist (S.H.R.) using World Health Organization diagnostic criteria. This study was approved by the Western Institutional Review Board and includes a waiver of informed consent and a HIPAA waiver of authorization.

Cohort specimens underwent sequential sequencing using ≥50 ng of DNA extracted from 661 formalin-fixed, paraffin-embedded (FFPE) clinical specimens. NGS was performed using hybridization-captured, adaptor-ligation libraries to a high, uniform coverage (mean coverage: 789.9x), covering 324 common oncogenes, and the intronic regions for 28 genes involved in rearrangement events. NGS data was analyzed for clinically relevant genomic alterations, including base pair substitutions, insertions/deletions, copy number alterations, and rearrangements/fusions. Sample inclusion was contingent on testing negative for 1p/19q co-deletion. Specimen calls for mutational signatures were made using the methodology as described in Zehir, et al. (2017), “Mutational Landscape of Metastatic Cancer Revealed From Prospective Clinical Sequencing of 10,000 Patients”, Nature Medicine 23:703-713. TMB was calculated based on ≤1.2 megabase (Mb), defined as the number of somatic, coding point mutations and indels per Mb of genomic material. Microsatellite Instability (MSI) status was determined for each tumor and reported as MSI stable (MSS), Ambiguous (MSA), and MSI High (MSI-H).

Latent Class Analysis: To identify latent molecular classes of yaGBMs, a latent class analysis was performed on a subset of yaGBMs (n=572), imposing the FDA TMB cutoff of 10 mut/Mb to reduce noise. A series of Gaussian Mixture Models were employed, varying the number of Gaussian Mixtures between 5 and 20. Model choice was performed using each mixture's Calinski-Harabasz score, which describes how well intragroup variance is minimized and intergroup variance is maximized. Using this metric, the first local maxima was determined to be the presented model.

### Results

Patient Demographics: CGP was performed on a cohort of 661 yaGBM specimens (≥18-≤40 yo). The cohort median (mean) age was 33 yo (32 yo); distributed among the following age ranges: ≥18-≤19 (n=16; 2.4%), ≥20-≤24 (n=82; 12.4%), ≥25-≤29 (n=121; 18.3%), ≥30-≤34 (n=157; 23.8%), and ≥35-≤40 years (n=285; 43.1%). Of these yaGBM patients, 62.0% (410/661) were male and 38.0% (251/661) were female (Table 1).

TABLE 1
Patient cohort demographics (age and sex).

| | Characteristic | Count | Percentage |
|---|---|---|---|
| Age | 18-19 | 16 | 2.4% |
| (N = 661) | 20-24 | 82 | 12.4% |
| | 25-29 | 121 | 18.3% |
| | 30-34 | 157 | 23.8% |
| | 35-40 | 285 | 43.1% |
| | Mean Age | 32 | |
| | Median Age | 33 | |
| Sex | Male | 410 | 62.0% |
| (N = 661) | Female | 251 | 38.0% |

Clinicopathological features were abstracted from pathology reports, when available. Patient recurrence status (de novo vs recurrent) was available in 89.5% (592/661) of samples; of which 58.1% (344/592) were identified as de novo and 41.9% (248/592) were identified as recurrent. MGMT promoter methylation status was available for 32.2% (213/661) of samples, of which 41.7% (89/213) were reported as methylated. Biopsy site data was available for 89.3% (590/661) of cases (Table 2).

TABLE 2
Patient cohort demographics (disease status
and MGMT promoter methylation status).

| | Characteristic | Count | Percentage |
|---|---|---|---|
| Disease Status | Primary | 344 | 58.1% |
| (N = 592) | Recurrent | 248 | 41.9% |
| MGMT Status | Methylated | 89 | 13.5% |
| (N = 213) | Unmethylated | 124 | 18.8% |
| Biopsy Site | Data Available | 590 | 89.3% |
| (N = 661) | | | |

Mutational signatures were determined in 79.6% (526/661) yaGBMs, of which 15.9% (84/526) presented with a dominant mutational signature. Alkylating signatures were used as a proxy for recurrence after standard-of-care treatment with temozolomide and/or nitrosoureas. Alkylating mutational signatures were detected in 6.8% (36/526) of cases whereas MMR, POLE and BRCA mutational signatures were detected in 5.9% (31/526), 0.6% (3/526) and 0.6% (3/526) of cases, respectively.

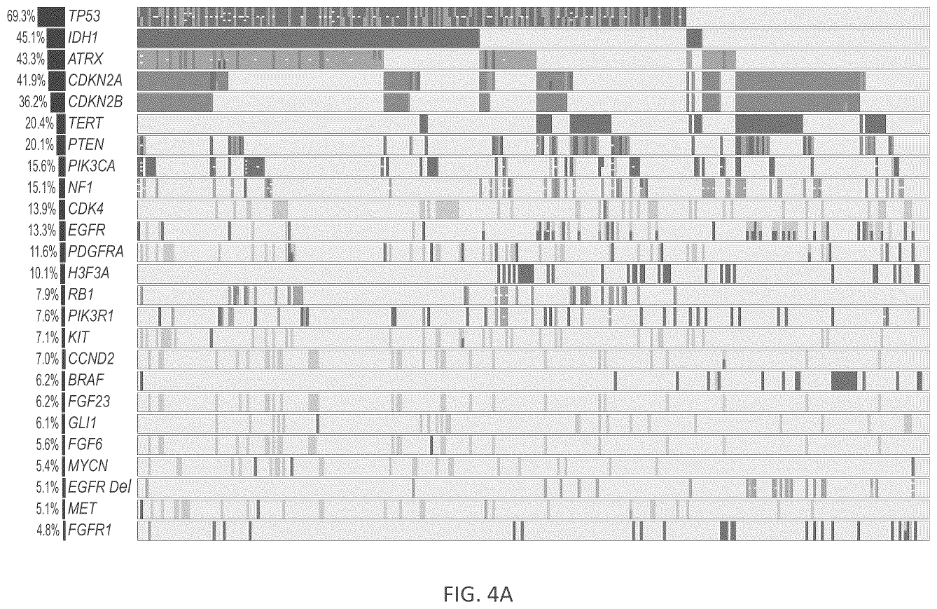

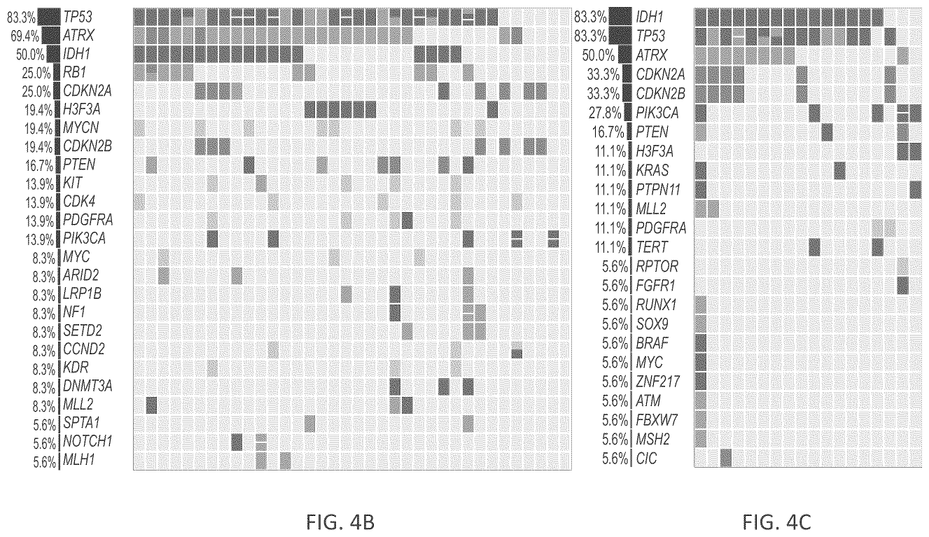

Genomic landscape of yaGBMs: Genomic alterations (GAs) were identified in almost all (660/661) yaGBM specimens, with an average 7.1 GAs per specimen. Commonly altered genes included TP53 (69.3%, 458/661), IDH1 (45.1%, 298/661), and ATRX (43.3%, 286/661); a finding consistent with previous reports demonstrating frequent mutations in these genes in adult lower grade gliomas. Other frequently altered genes included CDKN2A (41.9%, 277/661), CDKN2B (36.2%, 239/661), TERT promoter (20.4%, 135/661), PTEN (20.1%, 133/661), PIK3CA (15.6%, 103/661), NF1 (15.1%, 100/661), and CDK4 (13.9%, 92/661)—see FIG. 4A which shows a heatmap plot of color-coded mutation types for a plurality of biomarkers (rows) and patients (columns). Mutations in IDH2 were detected in rare cases (0.6%, 4/661).

A subset of yaGBMs showed histologic features of GBM variants including GBM with primitive neuroectodermal tumor features (GBM-PNET, n=36), GBM with oligodendroglial features (GBM-O, n=18) and gliosarcoma (GS, n=12). While IDH1, TP53 and ATRX mutations were enriched for across all GBM subtypes, H3F3A and MYCN alterations were detected in 19.4% (7/36) and 19.4% (7/36) of GBM-PNETs, respectively (FIG. 4B). Among GBM-Os, 15/18 (83.3%) were positive for mutations involving IDH1 (FIG. 4C) in contrast to GS which showed only 2/12 (16.7%) cases were IDH1 mutant.

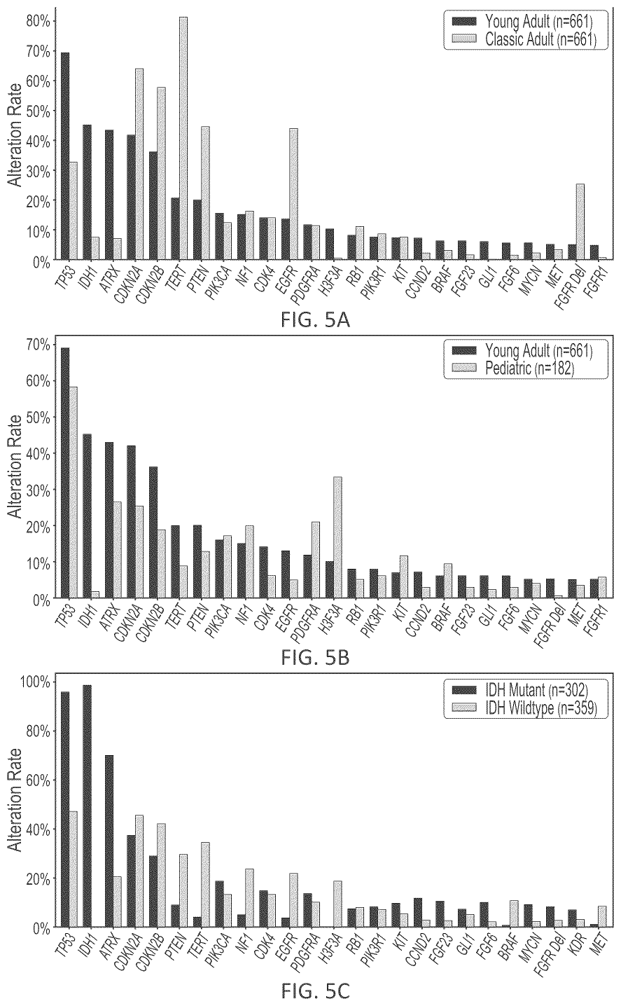

yaGBMs show distinct genomic profiles compared to pediatric and classic adult GBMs: Although the diagnostic classification of gliomas, including GBM, is based on the absence or presence of microscopic features established by WHO guidelines, we sought to determine whether the genomic profile of yaGBM is significantly different compared to classic adult GBMs (age >40 years) and pediatric GBMs (age <18 years). The frequencies of genomic alterations in the cohort of yaGBMs were compared to the frequencies of genomic alterations in a cohort of 661 randomly selected classic adult GBM samples interrogated by the same genomic assay. Similarly, the genomic landscape of the yaGBM cohort was compared to the genomic landscape of a cohort of 182 consecutively tested pediatric GBMs using the same assay (FIGS. 5A-5C).

A comparison of mutation rates of the 25 most frequently altered genes in yaGBMs was performed between classic adult GBMs and pediatric GBMs using a series of Fisher Exact tests with Holm-Bonferroni Correction. yaGBMs showed distinct genomic profiles compared to classic adult samples, with 15/25 genes significantly enriched in yaGBMs including TP53, IDH1, and other transcription factor, chromatin modifiers, cell cycle regulators and fibroblast growth factor pathway activators (FIG. 5A). Conversely, classic adult GBMs demonstrated significant enrichment for alterations in CDKN2A, CDKN2B, TERT-promoter, PTEN, and EGFR. Significant differences in the rate of EGFR intragenic deletions were also noted (e.g., EGFRvIII/vII) (FIG. 5A).

In comparing yaGBM and pediatric GBMs, 9/20 genes demonstrated significantly different mutation rates with the yaGBMs enriching for GAs in similar gene signatures described above, while pediatric GBMs showed significant enrichment for genomic alterations in H3F3A (33.5% vs 10.1%) and PDGFRA (20.9% vs 11.6%) (FIG. 5B).

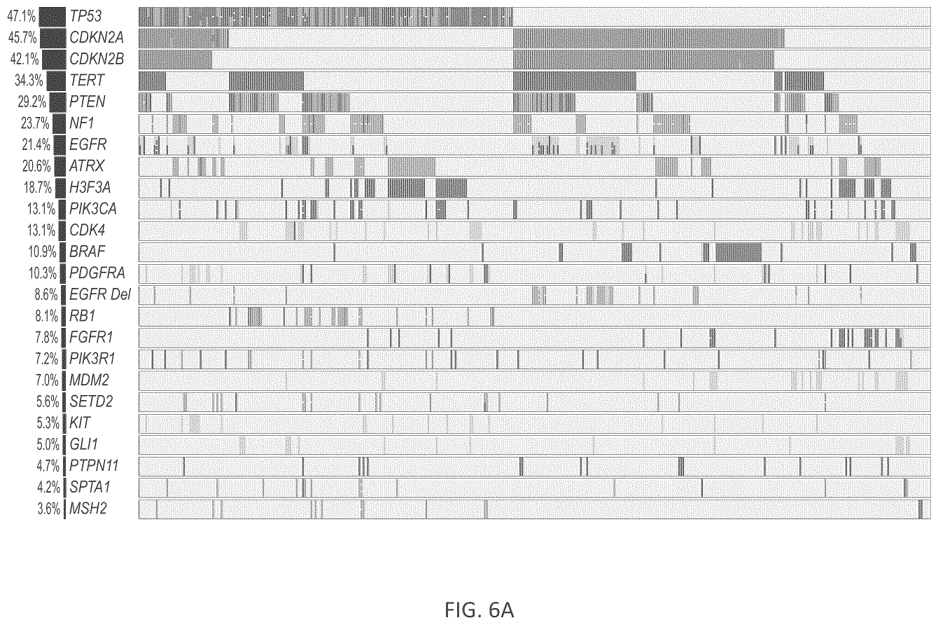

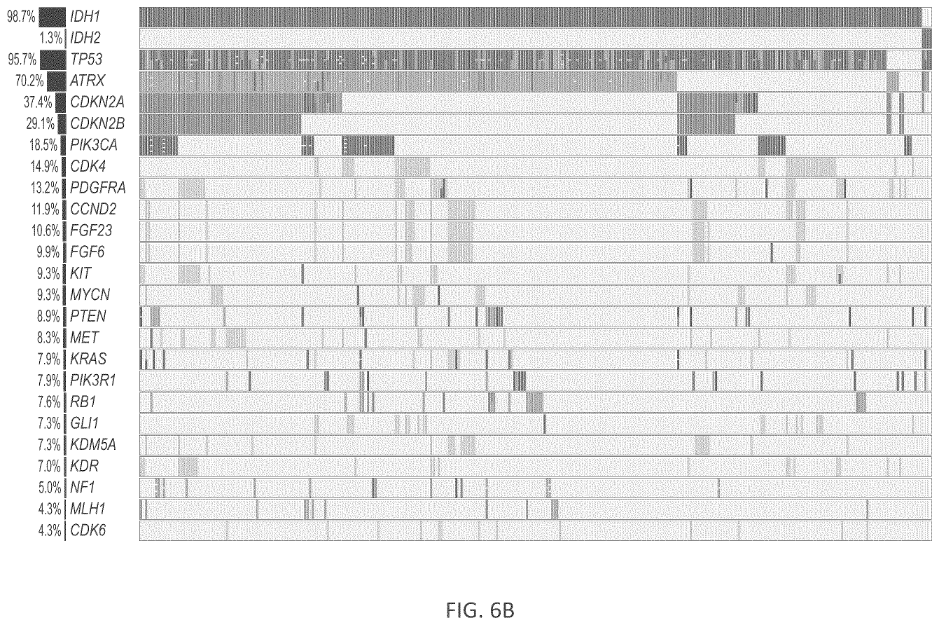

IDH1/2mut yaGBMs show distinct genomic profiles compared to IDH1/2 wt yaGBMs: yaGBMs can be broadly divided into IDH1/2 mutant and wild type groups; however, whether other genes show significantly different mutation rates remains unclear. To address this question, the mutation rates of the 25 most frequently altered genes in yaGBM with (IDH1/2mut) (n=302) were compared to those in yaGBM without IDH1/2 mutations (IDH1/2 wt) (n=359) (FIG. 6A and FIG. 6B). Alteration frequencies were significantly different in twelve (12/25) genes between the IDH1/2mut and IDH1/2 wt groups. Although TP53 was the most commonly altered gene in both cohorts, it showed significant enrichment in IDH1/2mut yaGBMs (95.7% vs 47.1%). Genes typical to the molecular presentation of GBMs, including TERT promoter, PTEN and EGFR were more frequently altered in IDH1/2 wt yaGBMs compared to IDH1/2mut cases. H3F3A alterations were also more frequent GAs in ATRX, MET, FGF, CCND2 and MYCN were enriched in IDH1/2mut cases, highlighting the difference in genomic landscapes between histologically indistinct tumors (FIG. 5C).

Among the 298 IDH1 mut yaGBMs, R132H accounted for 85.9% (256/298) of variants; others included R132G (15/298, 5.0%), R132C (13/298, 4.4%), R132S (12/298, 4.0%), and R132L (2/298, 0.7%). Four (4/661) yaGBMs with IDH2 mutations were identified (R140Q, R172K, R172S, R172M alterations). These findings highlight that approximately 14% of IDH1/2mut yaGBMs would not be detected by routine IDH1 R132H immune-histochemical methods.

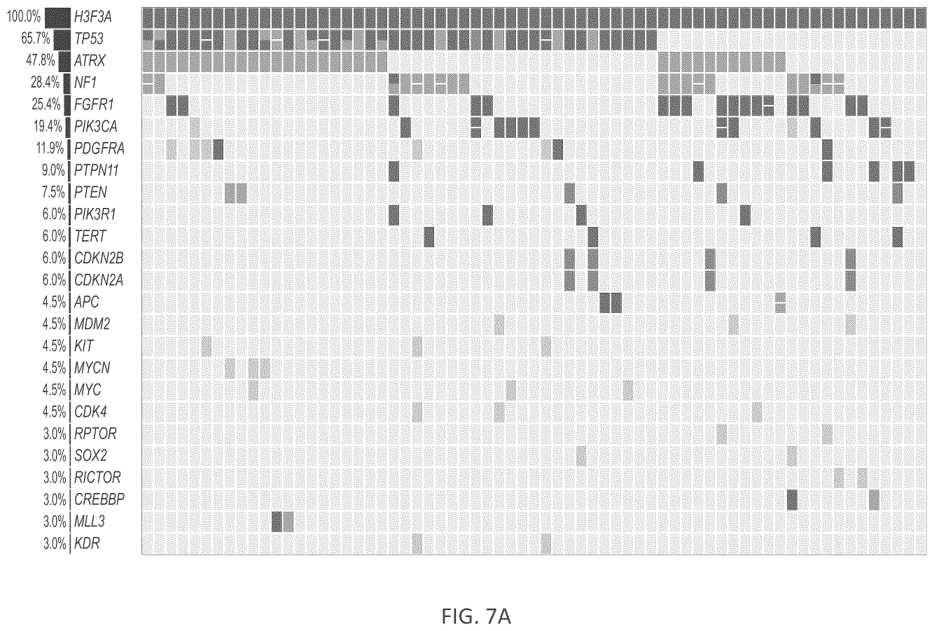

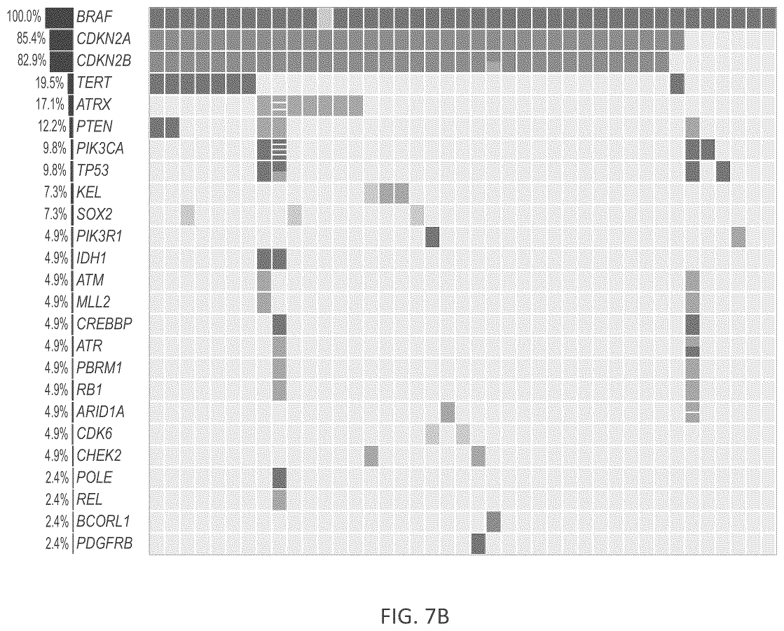

Recurrent H3F3A and BRAF mutations in IDH1/2 wt yaGBMs: A sub-analysis of yaGBMs with H3F3A mutations (n=67) showed that the majority (65.7%, 44/67) of cases harbored the K28M variant, followed by the G35R variant (29.9%, 20/67), and less frequently the G35V variant (4.5%, 3/67). All H3F3A mutant yaGBMs were IDH1/2 wt and had frequent co-occurring alterations in TP53 (44/67), ATRX (32/67), NF1 (19/67), and FGFR1 (17/67) (FIG. 7A).

Tumor anatomic location information was available (abstracted from pathology reports) for a subset (n=60) of H3F3A mutant samples and categorized into three groups: hemispheric (n=29), midline (n=23) and spinal cord (n=8). Of the 41 H3F3AK28M mutant tumors with anatomic site data, 75.6% (31/41) presented in the midline or spinal cord, and 24.4% (10/41) presented as hemispheric. Of the 17 H3F3AG35R and two G35V mutant samples with anatomic site data, all (19/19) were hemispheric.

BRAF alterations were identified in 6.2% (41/661) of yaGBMs, with V600E identified as the most common variant detected (82.9%, 34/41). Seven additional BRAF variants detected in individual cases included SNVs (D594G, K601E, D587G, G466E), insertions (R506_K507insVLR, V600_W604>DG) and a single case of amplification. Frequently co-altered genes among the BRAFmut cohort are CDKN2A (35/41), CDKN2B (34/41), TERT promoter (8/41), and ATRX (7/41) (FIG. 7B).

Recurrent gene fusions are rare in yaGBM: While driver gene fusions are reported with increased frequency in pediatric gliomas (e.g. KIAA1549-BRAF), it has not been determined whether fusions are significant drivers in yaGBMs. In this cohort, gene fusions were identified in 2.9% (19/661) of cases including FGFR3-TACC3 (n=3), FGFR1-TACC1 (n=2), KIAA1549-BRAF (n=2), ROS1-GOPC (n=2), EGFR-SEPT4 (n=4), RET-CCDC6 (n=2), FGFR2-GKAP1 (n=1), ATR-CPNE4 (n=1), ASTN2-ERG (n=1), and ARHGEF2-NTRK1 (n=1).

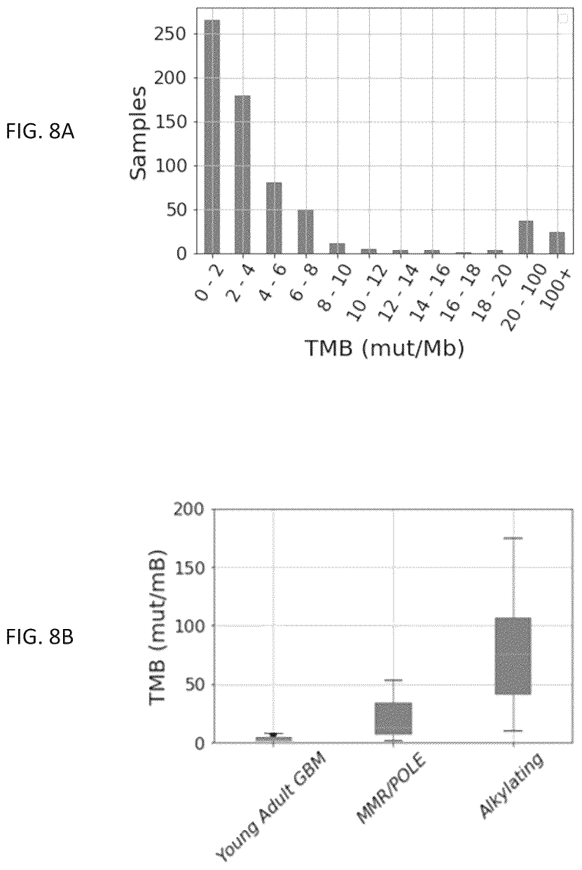

A subpopulation of hyper-mutated yaGBM harbors mutations in DNA mismatch-repair genes: Tumor mutational burden (TMB, mutations/Mb) was determined on up to 1.2 megabases of sequenced DNA, and TMB≥10 mutations/Mb was considered high TMB (TMB-high) per the US FDA approval of pembrolizumab for treatment of solid tumors. Of the yaGBM specimens with evaluable TMB scores (643/661), specimens typically showed TMB-low character. Only 11.0% (71/643) reported ≥10 mutations/Mb (FIG. 8A). Among TMB-high patients, 33.8% (24/71) were found to be ultra-mutated, which was defined as having a TMB≥100 mutations/Mb. Ultra-mutated tumors have been frequently associated with mismatch repair (MMR) or POLE mutations. Among patients with MMR/POLE mutational signatures (5.3%, 35/661), ultra-mutation was detected in 14.3% (5/35) patients (FIG. 8B). Similarly, patients with alkylating mutational signatures (36/661) also showed enrichment for ultra-mutator phenotype (33%, 12/36).

Tumors classified as TMB-high (n=71) frequently harbored mutations in TP53 (68/71), ATRX (52/71), IDH1 (40/71) and CDKN2A (34/71). Among TMB-high yaGBMs, 63.9% (45/71) harbored functional mutations in MMR or proofreading genes [MSH6, MLH1, MSH2, POLE1, and PMS2], while MMR genes were not present in any TMB-low specimens. Among the 40 IDH1/2mut TMB-H tumors, 70% (28/40) harbored mutations in MMR genes.

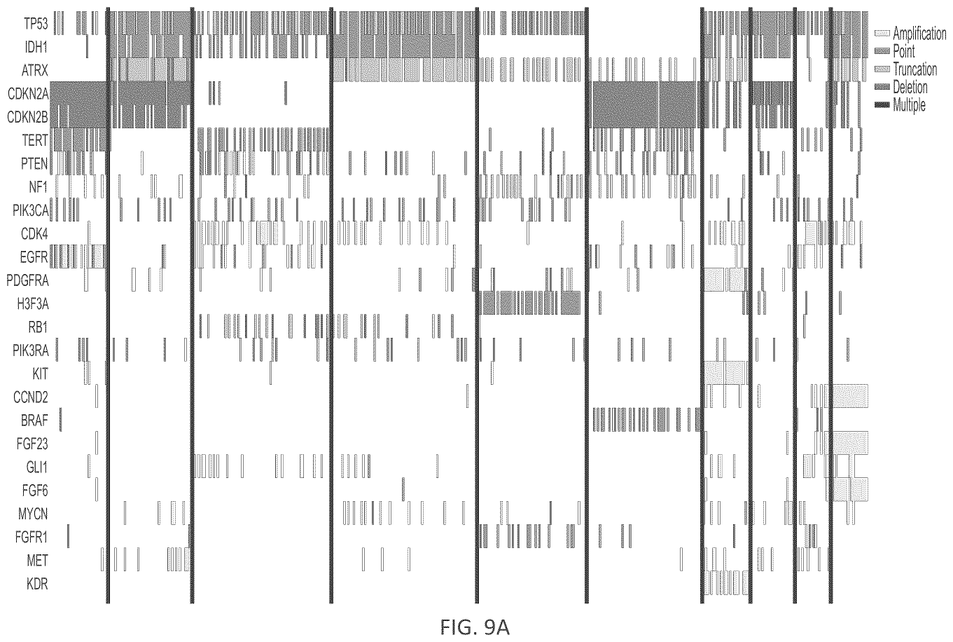

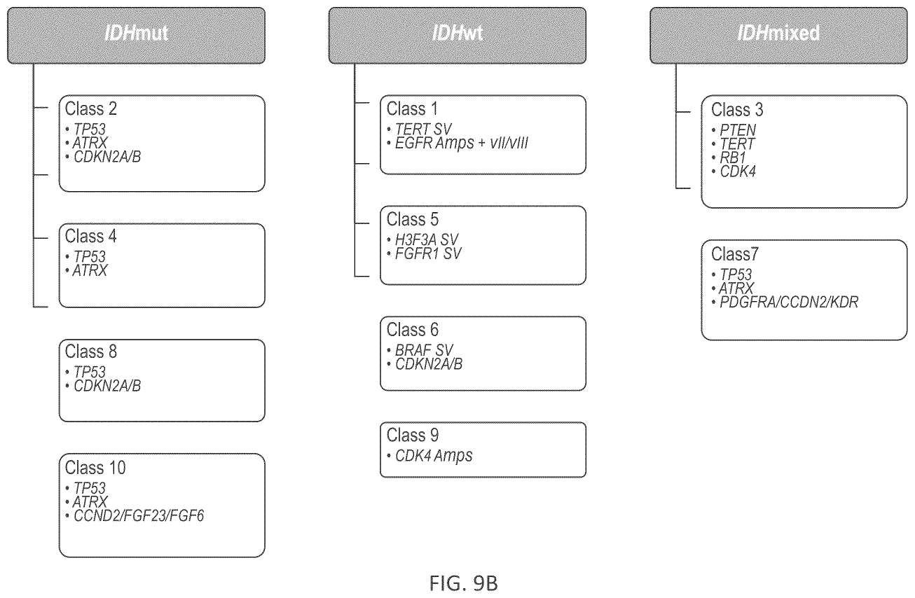

Latent Class Analysis Reveals Ten Distinct Genomic Subtypes of yaGBMs: Subtyping GBMs based on histology features is well established with recognized variants (per 2016 WHO Classification of CNS Tumors) including GBM with oligodendroglial features (GBM-O), gliosarcoma (GS) and GBM with primitive neuroectodermal tumor features (GBM-PNET), among others. Most tumors however show a more typical glial appearance that cannot be further sub-classified based on tumor cell morphology. Determining how histologically similar tumors differ genetically can improve the overall understanding of gliomagenesis and potentially guide novel treatment strategies. Using the methodology previously described, ten distinct molecular classes of yaGBMs were identified (FIG. 9A and FIG. 9B). FIG. 9A provides a heatmap plot of color-coded mutation types identified for specific genes (rows) in the ten distinct molecular classes (columns), arranged with Class 1 shown on the left and proceeding to Class 10 shown on the right.

As illustrated in FIG. 9B, IDHmut tumors were distributed among class 2, 4, 8 and 10. Class 2 (n=56) enriched for alterations in TP53, IDH1, ATRX, CDKN2A/B co-deletion, whereas Class 4 (n=98) only enriched for TP53, IDH1 and ATRX. Class 8 (n=30) showed enrichment for alterations in TP53, IDH1, and CDKN2A/B co-deletion. Class 10 (n=30) was primarily characterized by TP53, ATRX and IDH1 alterations; with co-amplifications of CCND2, FGFR23, FGFR6 and CDK4.

As illustrated in FIG. 9B, IDHWt tumors were distributed among class 1, 5, 6 and 9. Class 1 (n=44) showed enrichment for EGFR amplifications and co-occurring internal deletion events (EGFR vII/vIII). This was accompanied by CDKN2A/B codeletion events and TERT alterations. Class 5 (n=77) enriched for H3F3A mutant tumors whereas Class 6 (n=81) was characterized by alterations in BRAF with CDKN2A/B codeletion. Class 9 (n=21) showed amplification to CDK4, FGFR1, and GLII.

As illustrated in FIG. 9B, class 3 and 7 were composed of mixtures of IDH mutant and wild-type tumors. Class 3 (n=99) enriched for CDK4 amplifications as well as TERT promoter, PTEN and RB1 alterations. Class 7 (n=30) was characterized by co-amplification to PDGFRA, KIT and KDR. CDK4 amplification and CDKN2A/B co-deletion were also present. Alterations to IDH1 and ATRX were common as well.

### DISCUSSION

GBMs occur at all ages but can broadly be divided into three age groups encompassing pediatric (<18 yrs), young adult (18 to ≤40 yrs) and classic adult GBMs (>40 yrs) [11]. In this study, a retrospective analysis was performed of 661 yaGBMs (diagnosed using 2016 WHO classification criteria) that underwent CGP to characterize the genomic landscape of this unique and understudied patient population. To exclude the possibility of misclassified oligodendrogliomas confounding the analysis, all tumors included in the yaGBM study cohort were confirmed to be computationally negative for 1p/19q co-deletion, the biomarker that defines oligodendroglial lineage.

The study revealed that 45.1% of yaGBMs were IDH1 mutant with rare (0.6%) cases harboring mutations in IDH2. IDH mutant yaGBMs enriched for mutations in TP53 and ATRX, consistent with prior studies highlighting this recurrent genomic signature in adult lower grade astrocytomas. CDKN2A/B deletions were detected in 37.4% and 29.1% of IDH mutant and wild-type yaGBMs, respectively. Comparisons of frequently altered genes in yaGBMs with pediatric GBMs revealed striking differences including yaGBM enrichment for alterations in IDH1, ATRX, CDKN2A/B, pTERT and EGFR whereas pGBM enriched for H3F3A and PDGFRA alterations. Similarly, when comparing yaGBM to classic adult GBM, yaGBMs enriched for alterations in IDH1, TP53, ATRX, H3F3A, CCND2, FGF23, GLI1, FGF6 and FGFR1 while adult GBMs enriched for EGFR, PTEN, CDKN2A/B, and pTERT alterations.

To establish a genomic framework for sub-classifying yaGBMs, unsupervised machine learning methods, specifically LCA, were used as an objective way to identify genomic subpopulations within yaGBMs. LCA revealed 10 latent classes of yaGBMs based on pathogenic variants. IDH1/2^{mut} status stratified molecular classification; with three classes showing computed means>80% (Classes 2, 4, 8, and 10) and four showing computed means<20% (Classes 1, 5, 6, and 9).

Among IDH1/2^{mut} classes (Classes 2, 4, 8, and 10), enrichment for pathogenic mutations in both TP53 and ATRX were frequent, a combination that has been shown to lead gliomagenesis by suppression of SOX2. Classes 2, 4 and 8 showed similar features, stratifying primarily based on CDKN2A/B and ATRX alteration status. Functionally, loss of CDKN2A has been linked to faster tumor growth and confers a poorer overall prognosis for glioma patients. Additionally, Class 10 showed CDK4 amplification and co-amplification of CCND2, FGFR23, FGF6, which have shown susceptibility to alternative therapies employing CDK4/6 inhibitors, such as Abemaciclib. Ongoing clinical trials are exploring the use of CDK4/6 inhibitors in GBMs, either as monotherapy or in combination with immunotherapeutic strategies (NCT02981940, NCT04118036).

PTEN and pTERT showed significant enrichment among a number of classes (Class 1, 3, 6). PTEN mutations, often co-occurring with pTERT mutations, interact through the PTEN-AKT pathway to produce heightened cell proliferation. In GBMs, TERT promoter mutations carry a significantly worse overall survival (81.7 vs. 152.6 weeks; P=0.026). Targeted therapies for TERT are in early stages, but Eribulin shows promise in early trials of GBM mouse models.

Class 5 showed significant enrichment of H3F3A point mutations, primarily occurring as K28 and G35 alterations; sometimes accompanied by FGFR1 alterations. These have been previously described as showing epigenetically distinct populations.

Class 6 was defined primarily by BRAF V600E alterations, along with CDKN2A/B codeletion and PTEN/TERT mutants. Initial clinical data have shown some efficacy of combination BRAF (dabrafenib) and MEK (trametinib) inhibitors to slow disease progression.

Classes 7 and 10 appeared to be defined by the existence of CNAs, implicating large-scale chromosomal amplification events. These have been described previously in Zhang et al. (2016), “Co-Expression Modules of NF1, PTEN and SPROUTY Enable Distinction of Adult Diffuse Gliomas According to Pathway Activities of Receptor Tyrosine Kinases”, Oncotarget 7:59098-59114, which divided patients into RMPA high (RAS-RAF-MEK-MAPK pathway) and RMPAlow (PIK3-AKT pathway) subgroups. From our results, PDGFRA-KIT-KDR CNAs (Class 7) were associated with the RMPAhigh group and FGF6 & FGF23 CNAs (Class 10) were associated with the RMPAlow group, of which, gene-dosage dependent expression was confirmed in PDGFRA/KIT and FGF23, respectively. This distinction has clinical relevance, as RMPA low patients consistently show far better survival than their RMPA high counterparts (p<0.0001). Given that both groups show relatively high copy numbers (22.9 copies in FGF6/23, 24.0 in PDGFRA/KIT/KDR), these data indicate that these two amplicons may enhance gliomagenesis and define a unique genomic profile of yaGBMs.

Checkpoint inhibitor-based therapies (e.g., PD-L1 and PD-1 inhibitors) for GBM patients have advanced into clinical trials and initial studies in adult and pediatric GBM patients have shown profound anti-tumor responses in GBMs that harbor constitutional mismatch repair (MMR) gene mutations or germline POLE mutations. Elevated TMB is a biomarker associated with increased response to checkpoint inhibitors in peripheral solid tumors. Hyper-mutation in gliomas was recently explored in gliomas; with evidence showing this phenotype is likely driven by the emergence of sub-clonal MMR-mutant, TMZ resistant tumor populations during the course of therapy. Increased TMB often leads to a pattern of MMR mutations and a mutational signature unique to alkylating agents (TMZ) upon recurrence. MMR mutations, TMB, and mutational signature data were examined within this cohort to understand the landscape of hyper-mutation in yaGBMs. A bimodal distribution was identified, with 87.7% (572/661) presenting with a TMB<10 and 12.3% (89/661) presenting as TMBhigh (TMB≥10). These findings confirm that TMZ-associated hyper-mutation is a recurrent feature in a subset of yaGBMs and is frequently associated with underlying MMR gene mutations.

Taken together, these findings highlight that while GBMs may appear histologically similar across all age groups, the genomic drivers in each group vary significantly and these findings must be considered when designing and/or planning clinical trials.

### Exemplary Implementations

Exemplary implementations of the methods and systems described herein include:
1. A method comprising:
   providing a plurality of nucleic acid molecules obtained from a sample from each of a plurality of subjects;
   ligating one or more adapters onto one or more nucleic acid molecules from the plurality of nucleic acid molecules obtained from each sample;
   amplifying the one or more ligated nucleic acid molecules from the plurality of nucleic acid molecules obtained from each sample;
   capturing amplified nucleic acid molecules from the amplified nucleic acid molecules for each sample;
   sequencing, by a sequencer, the captured nucleic acid molecules for each sample to obtain a plurality of sequence reads that represent the captured nucleic acid molecules for each sample;
   receiving, at one or more processors, sequence read data for the plurality of sequence reads from each sample;
   receiving, at the one or more processors, subject data for a plurality of subjects diagnosed with the disease, wherein the subject data is based, at least in part, on the sequence read data for each sample and comprises an indication of whether a biomarker associated with a disease is present;
   obtaining, using the one or more processors, a plurality of candidate best fit models based on the subject data by:
      i) providing a predefined estimate of a number of subgroups;
      ii) generating a set of models based on the subject data, each model of the set comprising the same predefined estimate of the number of subgroups;
      iii) selecting a candidate best fit model from the set of models; and
      iv) repeating (i)-(iii) at least once using a different predefined estimate of the number of subgroups to obtain the plurality of candidate best fit models;
   selecting a best fit model from the plurality of candidate best fit models based on a fit statistic; and
   determining, using the one or more processors, a number of subgroups for the disease and an associated genomic profile for each subgroup based on the best fit model and the subject data.
2. The method of clause 1, further comprising determining to which disease subgroup an individual subject belongs based on the individual subject's data and the best fit model.
3. The method of clause 2, further comprising providing a treatment recommendation or outcome prediction for the individual subject based on the disease subgroup to which the individual subject belongs.
4. The method of any one of clauses 1 to 3, further comprising identifying a subgroup of subjects for participation in a clinical study based on a disease subgroup to which the subgroup of subjects belong.
5. The method of any one of clauses 1 to 4, further comprising identifying one or more driver mutations for a disease subgroup based on the associated genomic profile for the subgroup.
6. The method of any one of clauses 1 to 5, wherein the selecting of the best fit model is performed, at least in part, by the one or more processors.
7. The method of any one of clauses 1 to 5, wherein the selecting of the best fit model is performed, at least in part, by a user.
8. The method of any one of clauses 1 to 7, wherein the plurality of subjects have been diagnosed to have cancer.
9. The method of clause 8, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypercosinophilia, chronic cosinophilic leukemia, neuroendocrine cancers, or a carcinoid tumor.
10. The method of clause 8, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
11. The method of clause 9 or clause 10, further comprising treating the subject with an anti-cancer therapy.
12. The method of clause 11, wherein the anti-cancer therapy comprises a targeted anti-cancer therapy.
13. The method of clause 12, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Sccmblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimctinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), clotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligco), moxctumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
14. The method of any one of clauses 1 to 13, further comprising obtaining the sample from each subject of the plurality.
15. The method of any one of clauses 1 to 14, wherein the sample comprises a tissue biopsy sample, a liquid biopsy sample, or a normal control.
16. The method of clause 15, wherein the sample is a liquid biopsy sample and comprises blood, plasma, cerebrospinal fluid, sputum, stool, urine, or saliva.
17. The method of clause 15, wherein the sample is a liquid biopsy sample and comprises circulating tumor cells (CTCs).
18. The method of clause 15, wherein the sample is a liquid biopsy sample and comprises cell-free DNA (cfDNA), circulating tumor DNA (ctDNA), or any combination thereof.
19. The method of any one of clauses 1 to 18, wherein the plurality of nucleic acid molecules comprises a mixture of tumor nucleic acid molecules and non-tumor nucleic acid molecules.
20. The method of clause 19, wherein the tumor nucleic acid molecules are derived from a tumor portion of a heterogeneous tissue biopsy sample, and the non-tumor nucleic acid molecules are derived from a normal portion of the heterogeneous tissue biopsy sample.
21. The method of clause 19, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
22. The method of any one of clauses 1 to 21, wherein the one or more adapters comprise amplification primers, flow cell adaptor sequences, substrate adapter sequences, or sample index sequences.
23. The method of any one of clauses 1 to 22, wherein the captured nucleic acid molecules are captured from the amplified nucleic acid molecules by hybridization to one or more bait molecules.
24. The method of clause 23, wherein the one or more bait molecules comprise one or more nucleic acid molecules, each comprising a region that is complementary to a region of a captured nucleic acid molecule.
25. The method of any one of clauses 1 to 24, wherein amplifying nucleic acid molecules comprises performing a polymerase chain reaction (PCR) amplification technique, a non-PCR amplification technique, or an isothermal amplification technique.
26. The method of any one of clauses 1 to 25, wherein the sequencing comprises use of a massively parallel sequencing (MPS) technique, whole genome sequencing (WGS), whole exome sequencing, targeted sequencing, direct sequencing, or Sanger sequencing technique.
27. The method of clause 26, wherein the sequencing comprises massively parallel sequencing, and the massively parallel sequencing technique comprises next generation sequencing (NGS).
28. The method of any one of clauses 1 to 27, wherein the sequencer comprises a next generation sequencer.
29. The method of any one of clauses 1 to 28, wherein one or more of the plurality of sequencing reads overlap one or more gene loci within one or more subgenomic intervals in the sample.
30. The method of clause 29, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
31. The method of clause 29 or clause 30, wherein the one or more gene loci comprise ABL1, ACVR1B, AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CULAA, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRβ, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
32. The method of clause 29 or clause 30, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K8, PIGF, PTCH, RAF, RANKL, RET, ROS1, SLAMF7, VEGF, VEGFA, VEGFB, or any combination thereof.
33. The method of any one of clauses 1 to 32, further comprising generating, by the one or more processors, a report indicating the number of identified disease subgroups and an associated genomic profile for each subgroup.
34. The method of any one of clauses 2 to 33, further comprising generating, by the one or more processors, a report indicating the disease subgroup to which an individual subject belongs and an associated genomic profile for the individual subject.
35. The method of clause 33 or clause 34, further comprising transmitting the report to a healthcare provider.
36. The method of clause 35, wherein the report is transmitted via a computer network or a peer-to-peer connection.
37. A method for identifying a plurality of subgroups for a disease, the method comprising:
   receiving, at one or more processors, subject data for a plurality of subjects diagnosed with the disease, wherein the subject data comprises an indication of whether a biomarker associated with the disease is present;
   obtaining, using the one or more processors, a plurality of candidate best fit models based on the subject data by:
      i) providing a predefined estimate of a number of subgroups;
      ii) generating a set of models based on the subject data, each model of the set comprising the same predefined estimate of the number of subgroups;
      iii) selecting a candidate best fit model from the set of models; and
      iv) repeating (i)-(iii) at least once using a different predefined estimate of the number of subgroups to obtain the plurality of candidate best fit models;
   selecting a best fit model from the plurality of candidate best fit models based on a fit statistic; and
   determining, using the one or more processors, a number of subgroups for the disease and an associated genomic profile for each subgroup based on the best fit model and the subject data.
38. A method for identifying a plurality of subgroups for a disease, the method comprising: receiving, by at least one processor, subject data for a plurality of subjects diagnosed with the disease;
   obtaining, by the at least one processor, a plurality of candidate best fit models based on the subject data by:
      i) providing a predefined estimate of a number of subgroups;
      ii) generating a set of models based on the subject data, each model of the set comprising the same predefined estimate of the number of subgroups;
      iii) selecting a candidate best fit model from the set of models; and
      iv) repeating (i)-(iii) at least once using a different predefined estimate of the number of subgroups to obtain the plurality of candidate best fit models;
   selecting a best fit model from the plurality of candidate best fit models based on a fit statistic; and
   determining, using the one or more processors, a number of subgroups for the disease and an associated genomic profile for each subgroup based on the best fit model and the subject data.
39. The method of clause 37 or clause 38, wherein the selecting of the best fit model is performed, at least in part, by the one or more processors.
40. The method of clause 37 or clause 38, wherein the selecting of the best fit model is performed, at least in part, by a user.
41. The method of any one of clauses 37 to 40, further comprising determining to which disease subgroup an individual subject belongs based on the individual subject's data and the best fit model.
42. The method of clause 41, further comprising providing a treatment recommendation or outcome prediction for the individual subject based on the disease subgroup to which the individual subject belongs.
43. The method of any one of clauses 37 to 42, further comprising identifying a subgroup of subjects for participation in a clinical study based on a disease subgroup to which the subgroup of subjects belong.
44. The method of any one of clauses 37 to 43, further comprising identifying one or more driver mutations for a disease subgroup based on the associated genomic profile for the subgroup.
45. The method of any one of clauses 37 or clauses 38 to 44, wherein a biomarker associated with the disease comprises one or more genetic mutations.
46. The method of any one of clauses 37 to 45, further comprising modifying a specified panel of genes used for genomic profiling of the disease based on the identified number of subgroups for the disease and the associated genomic profile for each subgroup.
47. The method of clause 46, wherein modification of the specified panel of genes comprises modifying a bait set used to generate genomic profile data.
48. The method of any one of clauses 37 to 47, wherein the subject data for the plurality of subjects comprises genomic profile data.
49. The method of clause 48, wherein the subject data for the plurality of subjects further comprises data regarding subject sex, subject age, subject gender, subject height, subject weight, subject clinical history, subject sample type, or any combination thereof.
50. The method of clause 48 or clause 49, wherein the genomic profile data comprises a binary indication of whether a pathogenic mutation is present in each gene of a specified panel of genes.
51. The method of clause 50, wherein the genomic profile data is received in the form of a matrix comprising M rows×N columns, where each of the M rows comprises data for an occurrence of a genetic mutation in a given gene of the specified panel of genes in an individual subject, and each of the N columns comprises genetic mutation data across the specified panel of genes for an individual subject of the plurality of subjects.
52. The method of any one of clauses 37 to 51, wherein the plurality of candidate best fit models comprise non-probabilistic models.
53. The method of any one of clauses 37 to 51, wherein the plurality of candidate best fit models comprise probabilistic models.
54. The method of any one of clauses 37 to 53, wherein the predefined estimate of the number of subgroups includes a number of subgroups identified using a variational Bayesian method or agglomerative clustering method.
55. The method of clause 51 or clause 54, wherein the probabilistic models of a set of probabilistic models comprising the same predefined estimate of the number of subgroups are generated using different sets of initialization parameters.
56. The method of clause 55, wherein the different sets of initialization parameters comprise sets of randomly assigned initialization parameters.
57. The method of clause 55, wherein the different sets of initialization parameters comprise initialization parameters generated using a k-nearest neighbor (KNN) method.
58. The method of any one of clauses 37 to 57, wherein the candidate best fit model from the set of models comprising the same predefined estimate of the number of subgroups is selected based on optimization of an objective function.
59. The method of any one of clauses 37 to 58, wherein the candidate best fit model from the set of models comprising the same predefined estimate of the number of subgroups is selected based on optimization of a log-likelihood function.
60. The method of clause 59, wherein the selected candidate best fit model is the model which has a maximum log-likelihood value.
61. The method of any one of clauses 37 to 60, wherein the candidate best fit model is selected by the one or more processors at least in part based on a previous user selection.
62. The method of any one of clauses 37 to 61, wherein the candidate best fit model is selected by a machine learning model trained at least in part based on previous user selections.
63. The method of any one of clauses 37 to 62, further comprising specifying a set of one or more operational parameters for generating the set of models comprising the same predefined estimate of the number of subgroups, wherein the specified operational parameters comprise a maximum number of epochs to be performed, a tolerance on log-likelihood function for each epoch, a metric for calculation of covariance for each epoch, or any combination thereof.
64. The method of any one of clauses 37 to 63, wherein models of the set of models comprising the same predefined estimate of the number of subgroups are generated using a latent class analysis, clustering technique, or mixture modeling technique.
65. The method of clause 64, wherein the models are generated using a clustering technique, and wherein the clustering technique comprises a k-means clustering technique, a mean-shift clustering technique, a density-based spatial clustering of applications with noise (DBSCAN) technique, an agglomerative hierarchical clustering technique, a random forest technique, or any combination thereof.
66. The method of clause 65, wherein the clustering technique comprises a k-means clustering technique, and the output of the model comprises, for each subgroup (cluster), a centroid, an intra-cluster distance, an inertia value, an inter-cluster distance for each pair of clusters, a Dunn index, an expected prevalence for each cluster, or any combination thereof.
67. The method of clause 64, wherein the models are generated using a mixture model technique, and wherein the mixture modeling technique comprises a Gaussian mixture modeling technique, a multivariate Gaussian mixture modeling technique, a categorical mixture modeling technique, a variational Bayes mixture modeling technique, or any combination thereof.
68. The method of clause 67, wherein the mixture modeling technique comprises a Gaussian mixture modeling technique, and the output of the probabilistic model comprises, for each subgroup, a mean, μ, a covariance, Σ, and a mixing probability, π, an expected prevalence, or any combination thereof.
69. The method of clause 67 or clause 68, wherein determining optimal values for one or more model parameters comprises determining a maximum likelihood estimate of the one or more model parameters.
70. The method of any one of clauses 37 to 69, wherein the predefined estimate of the number of subgroups ranges from 1 to 100.
71. The method of any one of clauses 37 to 70, wherein the predefined estimate of the number of subgroups ranges from 1 to 50.
72. The method of any one of clauses 37 to 71, wherein the predefined estimate of the number of subgroups ranges from 1 to 20.
73. The method of any one of clauses 37 to 72, wherein the set of models generated for each predefined estimate of the number of subgroups comprises from 2 to 10 models.
74. The method of any one of clauses 37 to 73, wherein steps (i)-(iii) are repeated at least 2 times using a different predefined estimate of the number of subgroups each time.
75. The method of any one of clauses 37 to 74, wherein steps (i)-(iii) are repeated at least 5 times using a different predefined estimate of the number of subgroups each time.
76. The method of any one of clauses 37 to 75, wherein steps (i)-(iii) are repeated at least 10 times using a different predefined estimate of the number of subgroups each time.
77. The method of any one of clauses 37 to 76, wherein the fit statistic comprises an Akaike Information Criterion (AIC) score, a Bayesian Information Criterion (BIC) score, a Calinski-Harabasz (CH) score, or any combination thereof.
78. The method of any one of clauses 37 to 77, wherein the disease comprises a multifactorial inherited disorder.
79. The method of clause 78, wherein the multifactorial inherited disorder comprises heart disease, high blood pressure, Alzheimer's disease, arthritis, diabetes, cancer, obesity, or any combination thereof.
80. The method of any one of clauses 37 to 79, wherein the disease comprises a cancer.
81. The method of any one of clauses 46 to 80, wherein the specified panel of genes comprises between 10 and 100 genes for which there are known pathogenic or likely pathogenic mutations.
82. The method of any one of clauses 41 to 81, wherein the determination of to which disease subgroup an individual subject belongs is used to diagnose or confirm a diagnosis of disease in the subject.
83. The method of any one of clauses 80 to 82, further comprising selecting an anti-cancer therapy to administer to the subject based on the determination of to which disease subgroup the individual subject belongs.
84. The method of any one of clauses 80 to 83, further comprising determining an effective amount of an anti-cancer therapy to administer to the subject based on the determination of to which disease subgroup the individual subject belongs.
85. The method of clause 83 or clause 84, further comprising administering the anti-cancer therapy to the subject based on the determination of to which disease subgroup the individual subject belongs.
86. The method of any one of clauses 83 to 85, wherein the anti-cancer therapy comprises chemotherapy, radiation therapy, immunotherapy, a targeted therapy, or surgery.
87. A method for diagnosing a disease, the method comprising:
   diagnosing that a subject has the disease based on a determination of to which disease subgroup the subject belongs, wherein the disease subgroup is determined according to the method of any one of clauses 41 to 86.
88. A method for refining a diagnosis of disease, the method comprising:
   refining a diagnosis that a subject has the disease based on a determination of to which disease subgroup the subject belongs, wherein the disease subgroup is determined according to the method of any one of clauses 41 to 86.
89. A method of selecting an anti-cancer therapy, the method comprising:
   responsive to a determination of to which cancer subgroup a subject belongs based on an analysis of a sample from a subject, selecting an anti-cancer therapy for the subject, wherein the determination of cancer subgroup is made according to the method of any one of clauses 41 to 86.
90. A method of treating a cancer in a subject, comprising:
   responsive to a determination of to which cancer subgroup the subject belongs based on an analysis of a sample from the subject, administering an effective amount of an anti-cancer therapy to the subject, wherein the determination of cancer subgroup is made according to the method of any one of clauses 41 to 86.
91. A method for monitoring cancer progression or recurrence in a subject, the method comprising:
   determining to which cancer subgroup a subject belongs based on an analysis of a first sample obtained from the subject at a first time point according to the method of any one of clauses 41 to 86;
   determining to which cancer subgroup a subject belongs based on an analysis of a second sample obtained from the subject at a second time point; and comparing the first
   determination of cancer subgroup to the second determination of cancer subgroup, thereby monitoring the cancer progression or recurrence.
92. The method of clause 91, wherein the second determination of cancer subgroup is determined according to the method of any one of clauses 41 to 86.
93. The method of clause 91 or clause 92, further comprising selecting an anti-cancer therapy for the subject in response to the cancer progression.
94. The method of clause 91 or clause 92, further comprising administering an anti-cancer therapy to the subject in response to the cancer progression.
95. The method of clause 91 or clause 92, further comprising adjusting an anti-cancer therapy for the subject in response to the cancer progression.
96. The method of any one of clauses 93 to 95, further comprising adjusting a dosage of the anti-cancer therapy or selecting a different anti-cancer therapy in response to the cancer progression.
97. The method of clause 96, further comprising administering the adjusted anti-cancer therapy to the subject.
98. The method of any one of clauses 91 to 97, wherein the first time point is before the subject has been administered an anti-cancer therapy, and wherein the second time point is after the subject has been administered the anti-cancer therapy.
99. The method of any one of clauses 91 to 98, wherein the subject has a cancer, is at risk of having a cancer, is being routine tested for cancer, or is suspected of having a cancer.
100. The method of any one of clauses 91 to 99, wherein the cancer is a solid tumor.
101. The method of any one of clauses 91 to 99, wherein the cancer is a hematological cancer.
102. The method of any one of clauses 93 to 101, wherein the anti-cancer therapy comprises chemotherapy, radiation therapy, immunotherapy, a targeted therapy, or surgery.
103. The method of any one of clauses 37 to 86, further comprising determining to which disease subgroup an individual subject belongs based on an analysis of a sample obtained from the individual subject as a diagnostic value associated with the sample.
104. The method of any one of clauses 37 to 86, further comprising generating a genomic profile for the subject based on the determination of to which disease subgroup the subject belongs.
105. The method of clause 104, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
106. The method of clause 104 or clause 105, wherein the genomic profile for the subject further comprises results from a nucleic acid sequencing-based test.
107. The method of any one of clauses 104 to 106, further comprising selecting an anti-cancer therapy, administering an anti-cancer therapy, or applying an anti-cancer therapy to the subject based on the generated genomic profile.
108. The method of any one of clauses 41 to 86, wherein the determination of cancer subgroup for the sample is used in making suggested treatment decisions for the subject.
109. The method of any one of clauses 41 to 86, wherein the determination of cancer subgroup for the sample is used in applying or administering a treatment to the subject.
110. A system comprising:
   one or more processors; and
   a memory communicatively coupled to the one or more processors and configured to store instructions that, when executed by the one or more processors, cause the system to:
   receive subject data for a plurality of subjects diagnosed with a disease, wherein the subject data comprises an indication of whether a biomarker associated with the disease is present;
   obtain a plurality of candidate best fit models based on the subject data by:
      i) providing a predefined estimate of a number of subgroups;
      ii) generating a set of models based on the subject data, each model of the set comprising the same predefined estimate of the number of subgroups;
      iii) selecting a candidate best fit model from the set of models; and
      iv) repeating (i)-(iii) at least once using a different predefined estimate of the number of subgroups to obtain a plurality of candidate best fit models;
   select a best fit model from the plurality of candidate best fit models based on a fit statistic; and
   determine a number of subgroups for the disease and an associated genomic profile for each subgroup based on the best fit model and subject data.
111. The system of clause 110, further comprising instructions that, when executed by the one or more processors, cause the system to determine to which disease subgroup an individual subject belongs based on the individual subject's data and the best fit model.
112. The system of clause 111, further comprising instructions that, when executed by the one or more processors, cause the system to provide a treatment recommendation or outcome prediction for the individual subject based on the disease subgroup to which the individual subject belongs.
113. The system of any one of clauses 110 to 112, further comprising instructions that, when executed by the one or more processors, cause the system to identify a subgroup of subjects for participation in a clinical study based on a disease subgroup to which the subgroup of subjects belong.
114. The system of any one of clauses 110 to 113, further comprising instructions that, when executed by the one or more processors, cause the system to identify one or more driver mutations for a disease subgroup based on the associated genomic profile for the subgroup.
115. The system of any one of clauses 110 to 114, wherein the plurality of subjects have been diagnosed to have cancer.
116. A non-transitory computer-readable storage medium storing one or more programs, the one or more programs comprising instructions, which when executed by one or more processors of a system, cause the system to:
   receive subject data for a plurality of subjects diagnosed with a disease, wherein the subject data comprises an indication of whether a biomarker associated with the disease is present;
   obtain a plurality of candidate best fit models based on the subject data by:
      i) providing a predefined estimate of a number of subgroups;
      ii) generating a set of models based on the subject data, each model of the set comprising the same predefined estimate of the number of subgroups;
      iii) selecting a candidate best fit model from the set of models; and
      iv) repeating (i)-(iii) at least once using a different predefined estimate of the number of subgroups to obtain a plurality of candidate best fit models;
   select a best fit model from the plurality of candidate best fit models based on a fit statistic; and
   determine a number of subgroups for the disease and an associated genomic profile for each subgroup based on the best fit model and the subject data.
117. The non-transitory computer-readable storage medium of clause 116, further comprising instructions that, when executed by the one or more processors, cause the system to determine to which disease subgroup an individual subject belongs based on the individual subject's data and the best fit model.
118. The non-transitory computer-readable storage medium of clause 117, further comprising instructions that, when executed by the one or more processors, cause the system to provide a treatment recommendation or outcome prediction for the individual subject based on the disease subgroup to which the individual subject belongs.
119. The non-transitory computer-readable storage medium of any one of clauses 116 to 118, further comprising instructions that, when executed by the one or more processors, cause the system to identify a subgroup of subjects for participation in a clinical study based on a disease subgroup to which the subgroup of subjects belong.
120. The non-transitory computer-readable storage medium of any one of clauses 116 to 119, further comprising instructions that, when executed by the one or more processors, cause the system to identify one or more driver mutations for a disease subgroup based on the associated genomic profile for the subgroup.
121. The non-transitory computer-readable storage medium of any one of clauses 116 to 120, wherein the plurality of subjects have been diagnosed to have cancer.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method for identifying a plurality of subgroups for a disease, the method comprising:
receiving, by at least one processor, subject data for a plurality of subjects diagnosed with the disease;
obtaining, by the at least one processor, a plurality of candidate best fit models based on the subject data by:
i) providing a predefined estimate of a number of subgroups;
ii) generating a set of models based on the subject data, each model of the set comprising the same predefined estimate of the number of subgroups;
iii) selecting a candidate best fit model from the set of models; and
iv) repeating (i)-(iii) at least once using a different predefined estimate of the number of subgroups to obtain the plurality of candidate best fit models;
selecting a best fit model from the plurality of candidate best fit models based on a fit statistic; and
determining, using the one or more processors, a number of subgroups for the disease and an associated genomic profile for each subgroup based on the best fit model and the subject data.

2. The method of claim 1, further comprising determining to which disease subgroup an individual subject belongs based on the individual subject's data and the best fit model.

3. The method of claim 2, further comprising providing a treatment recommendation or outcome prediction for the individual subject based on the disease subgroup to which the individual subject belongs.

4. The method of claim 1, further comprising identifying a subgroup of subjects for participation in a clinical study based on a disease subgroup to which the subgroup of subjects belong.

5. The method of claim 1, further comprising identifying one or more driver mutations for a disease subgroup based on the associated genomic profile for the subgroup.

6. The method of claim 1, wherein a biomarker associated with the disease comprises one or more genetic mutations.

7. The method of claim 1, further comprising modifying a specified panel of genes used for genomic profiling of the disease based on the identified number of subgroups for the disease and the associated genomic profile for each subgroup.

8. The method of claim 1, wherein the subject data for the plurality of subjects comprises genomic profile data.

9. The method of claim 8, wherein the subject data for the plurality of subjects further comprises data regarding subject sex, subject age, subject gender, subject height, subject weight, subject clinical history, subject sample type, or any combination thereof.

10. The method of claim 1, wherein the predefined estimate of the number of subgroups includes a number of subgroups identified using a variational Bayesian method or agglomerative clustering method.

11. The method of claim 1, wherein the candidate best fit model from the set of models comprising the same predefined estimate of the number of subgroups is selected based on optimization of an objective function.

12. The method of claim 1, wherein the candidate best fit model from the set of models comprising the same predefined estimate of the number of subgroups is selected based on optimization of a log-likelihood function.

13. The method of claim 12, wherein the selected candidate best fit model is the model which has a maximum log-likelihood value.

14. The method of claim 1, wherein models of the set of models comprising the same predefined estimate of the number of subgroups are generated using a latent class analysis, clustering technique, or mixture modeling technique.

15. The method of claim 1, wherein the set of models generated for each predefined estimate of the number of subgroups comprises from 2 to 10 models.

16. The method of claim 1, wherein steps (i)-(iii) are repeated at least 2 times using a different predefined estimate of the number of subgroups each time.

17. The method of claim 1, wherein the fit statistic comprises an Akaike Information Criterion (AIC) score, a Bayesian Information Criterion (BIC) score, a Calinski-Harabasz (CH) score, or any combination thereof.

18. The method of claim 1, wherein the disease comprises a multifactorial inherited disorder or a cancer.

19. A system comprising:
one or more processors; and
a memory communicatively coupled to the one or more processors and configured to store instructions that, when executed by the one or more processors, cause the system to:
receive subject data for a plurality of subjects diagnosed with a disease;
obtain a plurality of candidate best fit models based on the subject data by:
i) providing a predefined estimate of a number of subgroups;
ii) generating a set of models based on the subject data, each model of the set comprising the same predefined estimate of the number of subgroups;
iii) selecting a candidate best fit model from the set of models; and
iv) repeating (i)-(iii) at least once using a different predefined estimate of the number of subgroups to obtain a plurality of candidate best fit models;
select a best fit model from the plurality of candidate best fit models based on a fit statistic; and
determine a number of subgroups for the disease and an associated genomic profile for each subgroup based on the best fit model and subject data.

20. A non-transitory computer-readable storage medium storing one or more programs, the one or more programs comprising instructions, which when executed by one or more processors of a system, cause the system to:
receive subject data for a plurality of subjects diagnosed with a disease;
obtain a plurality of candidate best fit models based on the subject data by:
i) providing a predefined estimate of a number of subgroups;
ii) generating a set of models based on the subject data, each model of the set comprising the same predefined estimate of the number of subgroups;
iii) selecting a candidate best fit model from the set of models; and
iv) repeating (i)-(iii) at least once using a different predefined estimate of the number of subgroups to obtain a plurality of candidate best fit models;
select a best fit model from the plurality of candidate best fit models based on a fit statistic; and
determine a number of subgroups for the disease and an associated genomic profile for each subgroup based on the best fit model and the subject data.
